(19) United States
(12) Patent Application Publication
(10) Pub. No.: US 2023/0105287 A1
(43) Pub. Date: Apr. 6, 2023

(54) SYSTEM AND METHOD FOR ORGAN INJECTION

(71) Applicant: DEKA Products Limited Partnership, Manchester, NH (US)

(72) Inventors: Hans E. JOHNSON, Salem, NH (US); Andrew G. REMEC, Manchester, NH (US); David D. B. CANNAN, Manchester, NH (US); Keith D. VIOLETTE, Sandown, NH (US); Matthew A. NORRIS, Londonderry, NH (US); Timothy D. MOREAU, Manchester, NH (US); Travis G. BIBEAU, Chester, NH (US); Margaret B. BRADLEY, Belgrade, ME (US); Stuart A. JACOBSON, Lexington, MA (US); Robert G. GLOEB-MCDONALD, Goffstown, NH (US); Scott R. BOWDEN, Pelham, NH (US); Richard E. ANDREWS, Manchester, NH (US); Dominic P. LOPRIORE, Amherst, NH (US); Alden S. NAEEM, Towson, MD (US); Trevor M. NARO, Nashua, NH (US); David A. SIMM, Westfield, MA (US)

(21) Appl. No.: 17/938,524

(22) Filed: Oct. 6, 2022

Related U.S. Application Data
(60) Provisional application No. 63/253,079, filed on Oct. 6, 2021.
(60) Provisional application No. 63/364,390, filed on May 9, 2022.

(51) Int. Cl. A61M 5/315; A61M 5/20; A61M 5/31; A61M 5/00

(52) U.S. Cl. CPC A61M 5/31578; A61M 5/2033; A61M 5/3129; A61M 5/001; A61M 2205/3331; A61M 2205/502; A61M 2202/09

(57) ABSTRACT

A system and method to enable treatment through cell therapy. The system can enable cell injection such as, for example, injecting beta cells/islets, cartilage cells, fat cells, and others. The system and method ensure that the delivery rate and delivered volume of the material pumped through a tube and into the injection needle is consistent over the course of the start-operation-stop process from run to run. The system and method can automatically stop delivery and alarm if an occlusion is detected during delivery.

### CROSS REFERENCE TO RELATED APPLICATIONS

This application claims the benefit of U.S. Provisional Patent Application Ser. No. 63/253,079 filed Oct. 6, 2021, entitled SYSTEM AND METHOD FOR ORGAN INJECTION (Attorney Docket No. AA707). This application also claims the benefit of U.S. Provisional Patent Application Ser. No. 63/364,390 filed May 9, 2022, entitled SYSTEM AND METHOD FOR ORGAN INJECTION (Attorney Docket No. AA831) both of which are incorporated herein by reference in its entirety.

### BACKGROUND

The present teachings relate generally to maintaining the health of an organ. The devices and methods of the present teachings are not limited in usefulness to one type of organ, as one of skill in the art will discover.

There is a need for a system to enable treatment through cell therapy. The system can enable cell injection such as, for example, injecting beta cells/islets, cartilage cells, fat cells, and others. Typically cells are injected as a mixture of cells in a medical liquid or cellular mixture. It is beneficial to ensure that the delivery rate of the material is consistent over the course of the start-operation-stop process from run to run. It is beneficial to adjust a pump rate according to the situation during the pumping operation. It is beneficial to include a user-friendly operation including, but not limited to, features such as quick fill of the injection needle, delivery accuracy, and end stop and occlusion detection.

### SUMMARY

The device of the present teachings solves the problems stated herein and other problems by one or a combination of the features stated herein.

One general aspect includes an apparatus to inject medical liquid. The apparatus also includes a housing; a syringe barrel with a fitting at the closed end, the syringe attached to the housing; a plunger including a lead screw, the plunger disposed in the syringe barrel a mechanism to drive the lead screw toward the closed end of the syringe barrel; an electric motor to drive the mechanism. The apparatus also includes where the mechanism is configured to be selectively disengaged from the lead screw and allowing the plunger to freely move axially in the syringe barrel.

Implementations may include one or more of the following features. The apparatus may include a second syringe that is configured to engage the fitting on the syringe barrel, where the second syringe may draw the plunger toward the closed end of the syringe barrel when the mechanism is selectively disengaged from the lead screw. The lead screw has at least one flat surface parallel to the lead screw axis. The lead screw cannot rotate and a second position where the lead screw is free to rotate; and where the rotation of the drive gear moves the lead screw along its axis when the anti-rotation flexure is in the first position and the lead screw can rotate and move long its axis when the drive gear is fixed and the anti-rotation flexure is in a second position. The key is configured to rotate in the housing and push the at least one arms away from the lead screw to put the anti-rotation flexure in the second position. The mechanism may include: a drive gear mechanically driven by the electric motor, the drive gear including a plurality of petal elements extending approximately on the axis of the of the lead screw, the petal elements have a thread surface that engages the lead screw in a first position and disengage from the lead screw in a second position. The plunger further includes a tip with a distal face that contacts the liquid and proximal surface that is detachable connected to the distal end of the lead screw, where the tip detaches from the lead screw when a negative pressure is applied to distal face. The plunger further includes a tip with a distal face that contacts the liquid and proximal surface that is configured to rotate relative to the lead screw. The electric motor may be displaced to disengage from the drive gear where lead screw may freely turn the drive gear as it moves axially. The apparatus may include a controller that receives input from a pressure sensor, at least one user input and controls the speed and direction of the electric motor. The at least one user input includes a trigger and a direction switch. The apparatus may include a battery switch The latch relay is closed by the first full pull of the trigger. The apparatus may include a battery connected to the controller. The apparatus may include status outputs may include at least one of an lcd display, a vibratory element and one or more status lights. The status light and lcd displays are controlled by the controller. The mechanism may include a reduction gear train on the the motor output shaft. The apparatus may include a rotation sensor that detects the number of rotations of an element in the mechanism. The apparatus where the rotation sensor measured rotations of a magnet on the end of the output shaft of the reduction gear train. The apparatus is disposable. The apparatus is only used for a single treatment. The housing, the drive mechanism, the plunger, the electric motor and syringe barrel are disposable after a single treatment. The housing, the drive mechanism, the plunger, the electric motor and syringe barrel are disposable are sterilized as a unit before use. The treatment kit is sterilized with ethylene oxide.

One general aspect includes a apparatus to inject medical liquid. The apparatus also includes a housing; a syringe barrel with a fitting at the closed end, the syringe attached to the housing; a plunger including a lead screw, the plunger disposed in the syringe barrel a mechanism to drive the lead screw toward the closed end of the syringe barrel; an electric motor to drive the mechanism; a controller that controls the electric motor speed; and a pressure sensor mounted in the housing and configured to measure the pressure of liquid in the syringe barrel, where the controller varies the electric motor speed based on a pressure signal received from the pressure sensor.

Implementations may include one or more of the following features. The apparatus where the pressure sensor is mounted to sense pressure in a tube downstream of the syringe. The pressure sensor is mounted in front of the syringe barrel to sense an axial force applied by plunger on the liquid in the barrel. The controller detects an occlusion when the pressure signal exceeds a predetermined value. The controller reverses the electric motor an occlusion is detected, the controller continues to run the electric motor in reverse until the pressure signal drops below a predetermined value. The controller monitors a parameter of the electric motor and the controller declares an end of stroke based on the motor parameter and the pressure signal. The controller reverses the electric motor when an end of stroke is declared, the controller continues to run the electric motor in reverse until the pressure signal drops below a predetermined value.

One general aspect includes a method to deliver a volume of a cells mixture to an organ in vivo with a disposable syringe pump including a syringe barrel fluidly connected to an insertion needle. The method also includes positioning the distal end of the insertion needle in the organ; receiving a user input to deliver the cellular mixture, driving the electric motor at a first speed receiving a signal from the pressure sensor, driving the electric motor at a second speed after the the signal from the pressure sensor exceeds a first predetermined value, monitor the number of rotation of an element in the mechanism, determine the delivered volume of cellular mixture, stopping the electric motor when the delivered volume exceed a predetermined volume, and reversing the electric motor until the pressure signal drops below a second predetermined value.

Implementations may include one or more of the following features. The method where the user input is a signal from a switch or trigger and the method may include the steps of: monitoring the user input signal; stopping the electric motor, when less than the predetermined volume has been delivered and the user signal ends provided; and restarting the electric motor when the user signal restarts. The user input is a signal from a switch or trigger and the method may include the steps of: ending the user input after the predetermined volume is delivered, reinitiating the user input, and delivering a second predetermined volume of the cellular mixture.

One general aspect includes a apparatus to inject medical liquid. The apparatus also includes a housing; a syringe barrel with a fitting at the closed end, the syringe attached to the housing; a plunger including a lead screw, the plunger disposed in the syringe barrel a mechanism to drive the lead screw toward the closed end of the syringe barrel; an electric motor to drive the mechanism; a pressure sensor configured to measure a pressure characteristic of a medical liquid pressure in the syringe barrel or a line fluidly connected to the syringe barrel; a rotation sensor configured to measure the rotations of an element of the mechanism; and a controller, where the controller determines the volume of medical liquid injected based on a pressure value and the number of rotations measured the rotations sensor.

Implementations may include one or more of the following features. The apparatus of where controller is configured to slow the electric motor after the pressure signal exceed a predetermined pressure and the controller calculates the volume of fluid injected based on the predetermined pressure. The predetermined pressure is 800 mmhg. Controller calculates the volume of fluid injected based on the average pressure during the previous injection of a predetermined volume of medical liquid. The pressure sensor is mounted to sense pressure in a tube downstream of the syringe. The pressure sensor is mounted in front of the syringe barrel to sense an axial force applied by plunger on the liquid in the barrel.

One general aspect includes a method for injecting an aliquot of material into an organ using a handheld injection apparatus. The method also includes filling the delivery component with the aliquot; activating the delivery mechanism; delivering, by the delivery mechanism, a pre-selected volume of the aliquot at a pre-selected rate; and detecting a status of the injection apparatus.

Implementations may include one or more of the following features. The method as where the delivery component may include: an aliquot container; an organ penetration device; and a plunger operably coupled with the aliquot container, the plunger pushing the aliquot towards the organ through the aliquot container into the organ penetration device. The delivery component may include: a disposable component. The delivery component may include: a durable component. The delivery mechanism may include: a spring, a plunger component of the delivery component, and a lead screw pushing the plunger component when the spring is released. The delivery mechanism may include: a lead screw; a motor; and a gear train driven by the motor, the gear train driving the lead screw, the lead screw activating the delivery component. The method as may include: detecting the status of the aliquot. The method as may include: detecting characteristics of the aliquot. The delivery mechanism may include: a mechanical component. The delivery mechanism may include: an electromechanical component. The injection apparatus may include: a mechanical delivery component operably coupled with an electromechanical delivery mechanism. The injection apparatus may include: a purely mechanical delivery component operably coupled with a mechanical delivery mechanism. Activating the delivery mechanism may include: winding a spring, the spring being operably coupled with the delivery mechanism. Activating the delivery mechanism may include: depressing a trigger, the trigger being operably coupled with a motor, the motor being operably coupled with the delivery mechanism. Delivering the aliquot may include: driving the delivery mechanism forward to deliver the aliquot. Detecting the status of the injection apparatus may include: raising a first alert if the status of the delivery mechanism meets at least one first pre-selected criterion, ceasing delivery of the aliquot, and driving the delivery mechanism in reverse. Detecting the status of the injection apparatus may include: raising a second alert if a rate of delivery of the aliquot meets at least one second pre-selected criterion, and driving the delivery mechanism in reverse. Detecting the status of the injection apparatus may include: raising a third alert if a pressure of the aliquot meets at least one third pre-selected criterion, and stopping the delivery mechanism. Detecting the status of the injection apparatus may include: activating indicator lights when at least one fourth pre-selected criterion is met.

One general aspect includes a handheld injection apparatus for injecting an aliquot of material into an organ. The handheld injection apparatus also includes a delivery mechanism. The apparatus also includes a delivery component operably coupled with the delivery mechanism, the delivery component including a container configured to hold the aliquot, where the delivery mechanism activates the delivery component to deliver a pre-selected volume of the aliquot to the organ at a pre-selected rate.

Implementations may include one or more of the following features. The handheld injection apparatus as where the material may include: cell therapy. The material may include: at least one medication. The material may include: at least one drug. The organ may include: a kidney. The organ may include: a liver. The delivery component may include: an organ penetration device; and a plunger operably coupled with the container, the plunger pushing the aliquot towards the organ through the container into the organ penetration device. The handheld injection apparatus as may include: at least one disposable component. The handheld injection apparatus as may include: at least one durable component. The delivery mechanism may include: a spring, a plunger component of the delivery component, and a lead screw pushing the plunger component when the spring is released. The delivery mechanism may include: a lead screw; a motor; and a gear train driven by the motor, the gear train driving the lead screw, the lead screw activating the delivery component. The handheld injection apparatus as may include: at least one sensor configured to detect at least one characteristic of the aliquot, and at least one processor configured to control the handheld injection apparatus based at least on the at least one characteristic. The handheld injection apparatus as may include: at least one sensor configured to detect at least one status value of the handheld injection device, and at least one processor configured to control the handheld injection apparatus based at least on the at least one status value. The handheld injection apparatus as may include: at least one sensor configured to detect at least one status value of the handheld injection device, and at least one processor configured to report the at least one status to a user. The handheld injection apparatus as may include: at least one sensor configured to detect at least one status value of the handheld injection device, and at least one processor configured to log the at least one status. The delivery mechanism may include: an electromechanical component. The delivery mechanism may include: a purely mechanical component. The handheld injection apparatus may include: a mechanical delivery component operably coupled with an electromechanical delivery mechanism. The handheld injection apparatus may include: a mechanical delivery component operably coupled with a mechanical delivery mechanism. The delivery mechanism may include: a spring, the spring being operably coupled with the delivery mechanism, the spring being configured to activate the delivery mechanism. The delivery mechanism may include: a motor operably coupled with the delivery mechanism; and a trigger, the trigger being operably coupled with the motor. The delivery mechanism may include: being configured to be driven forward to deliver the aliquot. The handheld injection apparatus as may include: a status of the handheld injection apparatus, and an alert system raising a first alert if the status of the handheld injection apparatus meets at least one first pre-selected criterion and driving the delivery mechanism in reverse. The handheld injection apparatus as may include: a status of the handheld injection apparatus, and an alert system raising a second alert if a rate of delivery of the aliquot meets at least one second pre-selected criterion and driving the delivery mechanism in reverse. The handheld injection apparatus as may include: a status of the handheld injection apparatus, and an alert system raising a third alert if a pressure of the aliquot meets at least one third pre-selected criterion and stopping the delivery mechanism. The handheld injection apparatus as may include: a status of the handheld injection apparatus, and an alert system activating indicator lights when at least one fourth pre-selected criterion is met. The handheld injection apparatus as may include: at least one pressure sensor configured to detect pressure of the aliquot. The handheld injection apparatus as may include: at least one pressure sensor integrated with the delivery mechanism, the at least one pressure sensor configured to detect pressure of the aliquot.

One general aspect includes an organ injection device for injecting substance into organs. The organ injection device also includes a motor; an activation means configured to activate the motor, a delivery mechanism configured to deliver the substance, an energy transfer mechanism configured to transfer the energy generated by the motor to the delivery mechanism, a controller configured to control the energy transfer mechanism, and at least one sensor configured to provide data about the motor and the delivery mechanism to the controller.

Implementations may include one or more of the following features. The injection device as may include: a stop mechanism configured to inhibit the delivery mechanism from delivering the substance. The injection device as may include: an energy storage means configured to power the motor. The injection device as may include: a monitor configured to provide information about the controller. The controller may include: an electromagnetic controller configured to control the speed of the motor. The injection device as may include: a brake configured to stop the delivery mechanism. The organ injection device as may include: a case configured to protect the motor, the activation means, the delivery mechanism, the energy transfer mechanism, the at least one sensor, and the controller from microcontaminants. The organ injection device as may include: a case configured to protect the motor, the activation means, the delivery mechanism, the energy transfer mechanism, the at least one sensor, and the controller from macrocontaminants. The organ injection device as may include: a delivery mechanism case configured to protect the delivery mechanism from environmental contaminants. The organ injection device as may include: a directional switch configured to enable multi-directional operation of the organ injection device. The organ injection device as may include: at least one status indicator. The organ injection device as may include: a first speed of the motor, the motor operating at the first speed when beginning to dispense the substance; a second speed of the motor, the motor operating at the second speed after the substance has begun to be dispensed; and a third speed of the motor, the motor operating at the third speed after at least one pre-selected condition has been met.

One general aspect includes a mechanical injector for injecting substance into an organ. The mechanical injector also includes a delivery mechanism; a trigger, and an activation mechanism configured to move the substance in the delivery mechanism into the organ when the trigger is engaged.

Implementations may include one or more of the following features. The mechanical injector as may include: a protective casing configured to enclose the injection device. The protective casing may include: a plurality of interconnecting sections. The protective casing may include: a single section. The protective casing may include: a body lid, a body upper operably coupled with the body lid, a mid-body operably coupled with the body upper, and a handle casing operably coupled with the mid body. The handle casing may include: a left side handle casing, and a right side handle casing operably coupled with the left side handle casing. The activation mechanism may include: a spring-wound apparatus with a timing escapement driving the delivery mechanism, the trigger controlling when the spring-wound apparatus is released to drive the delivery mechanism. The delivery mechanism may include: a syringe plunger, and a lead screw operably coupled with and driving the syringe plunger The delivery mechanism may include: a lead screw; a syringe stopper operably coupled with the lead screw; and an axially stable drive nut configured to drive the lead screw into a syringe plunger by rotating in place, the lead screw configured to be rotationally stable and translating forward. The drive nut may include: gear teeth around its exterior, the drive nut being operably coupled with a drive shaft via a gear train, the drive shaft operably coupled with a torque drum assembly, the torque drum assembly being wound with a spring, the spring configured with multiple turns to provide constant torque output from each of the multiple turns, where the great train is configured to be geared up or geared down to meet a pre-selected number of drive nut turns per volume of a stroke at the syringe plunger, and where the drive shaft is operably coupled to a timing mechanism ensuring that the drive shaft rotates at a fixed rate. The timing mechanism may include: a static rate. The timing mechanism may include: a gear shift configured to alter which gears connect to the drive shaft, enabling multiple fixed rates of delivery. The torque drum assembly may include: a torque drum; and a storage drum configured to hold a spring, where the activation mechanism is configured to be charged by rotating the torque drum and coiling the spring onto the torque drum. The activation mechanism may include: a movable assembly including a bolt rail; a ball knob configured to slide along the bolt rail; a primary pulley and a secondary pulley; a pulley belt operably coupling the primary pulley and the secondary pulley, where the ball knob, the primary pulley, the secondary pulley, and the pulley belt operate cooperatively with the storage drum to charge the activation mechanism. The activation mechanism may include: a slanted beam; a rotating arm including a tooth; and at least one torsion spring configured to hold the rotating arm at a center of a swing arc, where, when an aliquot is completed, the tooth contacts the slanted beam, the slanted beam pushing the rotating arm to compress one of the at least one torsion spring. The trigger may include: a spring-loaded mechanism that, at rest, pushes a wedge into teeth of the drive nut preventing rotation of the drive nut, the trigger, when depressed, pulling the wedge from the teeth without unwinding the spring-loaded mechanism. The mechanical injector as may include: a turning device configured to rewind the spring-loaded mechanism after a pre-selected number of aliquots of the substance have been delivered. The turning device may include: a ratcheting lever. The turning device may include: a linear slide with a gear rack configured to engage with a bevel gear on the drive shaft. The mechanical injector as may include: at least one sensor associated with the delivery mechanism, the at least one sensor configured to detect changes in characteristics of the delivery mechanism. The mechanical injector as may include: at least one sensor configured to monitor characteristics of the injector as sensor data; and at least one processor configured to receive the sensor data from at least one sensor, the at least one processor configured to store a buffer of the characteristics from a pre-selected amount of time at a pre-selected sampling rate, the at least one processor configured to compare values of the sensor data gathered at a first time to the values of the sensor data gathered at a second time different from the first time by the pre-selected amount of time, the at least one processor monitoring changes in the characteristics indicating that the delivery mechanism is incorrectly positioned, the at least one processor configured to raise an alert based on the changes in the characteristics. The mechanical injector as may include: a delivery mechanism restraint configured to secure the delivery mechanism against movement when under pressure by the activation mechanism. The activation mechanism may include: drawing a vacuum in the delivery mechanism, the vacuum pulling the substance into the delivery mechanism.

One general aspect includes the organ injection device as where the at least one status indicator may include: The organ injection device also includes a moving pattern of lights activated when the activation means is enabled.

Implementations may include one or more of the following features. The injection device as. The injection device includes a charging means configured to charge the energy storage means. The at least one status indicator may include: The organ injection device includes a delivery mechanism status indicator configured to provide a status of the delivery mechanism.

The present teachings relate generally to injection of cell therapies or other medications and drugs. Any version of the injection device can include features such as, but not limited, a sealed enclosure, an integrated syringe with a lead screw driving the plunger and volumetric markings, a pressure sensor that directly mates to the syringe, a motor and gearbox used to drive the lead screw, a toggle switch used to select the direction of movement and reset the deposit volume accumulator, a trigger switch used to initiate movement of the motor, a display used to convey the device's status, a vibration motor used to non-visually indicate that the user can check the display, a power supply used to power the device, a power supply switch used to externally engage the power supply to the system, a release mechanism to allow recovery of the injection solution in the event of a complete device failure, circuit boards, and onboard software used to, for example, but not limited to, control the injection device subsystems and log data.

The device of the present teachings can drive boluses of possibly viscous materials at specific rates from a disposable device whose features are monitored in order to maintain the rate and other characteristics of the delivery. In one exemplary treatment, organ cells are stored in DMSO. The DMSO/organ cell mixture or medical liquid is stored in a frozen condition and then thawed before being injected into a body. The device can be used to extract materials from vials, and a syringe can be integrated into the device. The device includes an automated way to inject a specific amount. The device can target a desired flow rate and flow volume, possibly increasing flow rate at the beginning and end of the injection. The device can detect changes in delivery pressure that can indicate the status of the injection, for example, due to occlusions, unintended target in the body or due unexpected pressure in the target organ.

A first exemplary device includes a trigger that is depressed for delivery, a syringe, a spring to drive the syringe, and an electrical box for a pressure sensor circuit. This primarily mechanical device is constructed to have a minimum number of points of failure, but yet be durable. Such a device can be manufactured economically and easy to operate. The device can optionally include a means for making a lead screw easily retractable, thereby removing the need to rewind the spring between aliquots.

The device can also optionally include another mechanism for winding the spring and a means for stopping delivery automatically after each aliquot. A stopcock can optionally be added to the trigger, along with mounting geometry for the pressure sensor.

A second exemplary device includes a means to adjust delivery rates/volumes, provide user feedback, remove the operator from a pressure feedback loop, supports vial decanting, and includes a digital stopcock. The second device includes a means for varying decanting speed, a means for tuning the digital stopcock, a means for tuning a rate controller, and a soft stop at actuator range extents. The second exemplary device includes a processor that controls a motor that controls a pump that is connected to a tube at one end of the tube, and to an injection needle at the other end of the tube. The injection needle pressure is monitored by a pressure sensor. The device is powered by a power supply such as, for example, but not limited to, a battery. Power supply characteristics can be monitored by the processor.

Any version of the injection device can include features such as, but not limited, a sealed enclosure, an integrated syringe with a lead screw integrated into the plunger and volumetric markings, a pressure sensor that directly mates to the syringe, a motor and gearbox used to drive the lead screw, a toggle switch used to select the direction of movement and reset the deposit volume accumulator, a trigger switch used to initiate movement of the motor, a display used to convey the device's status, a vibration motor used to non-visually indicate that the user can check the display, a power supply used to power the device, a power supply switch used to externally engage the power supply to the system, a release mechanism to allow recovery of the injection solution in the event of a complete device failure, circuit boards, and onboard software used to, for example, but not limited to, control the injection device subsystems and log data. In place of the syringe, a mechanical bellow/baffled cartridge or a diaphragm cartridge can be used.

The electromechanical component of the system of the present teachings is a disposable, pre-sterilized injection device, or a durable device with a single-use syringe inserted into it, which draws liquid into a syringe using a piston, then dispenses it at a controlled rate. In an aspect, the injection device includes an injection needle or other type of injection device, transfer tubing, and a vial adaptor. The injection needle can include a high gauge, non-coring (side-port) needle that acts as a semi-rigid liquid path injection deposit positioning aide. The transfer tubing acts as a flexible liquid path between the injection device and the injection needle to decrease the chances of accidently dislodging the injection needle while performing the injection. The vial adaptor is used to assist in filling the injection device with injection solution. The vial adaptor can pierce the vial septum and a vent to prevent vacuum from building up in the vial.

Components that are used by the system of the present teachings that are available commercially can include, but are not limited to including, disposables including an injection needle and a guide needle, transfer tubing, and a vial adapter. The guide needle or trocar is configured to pierce from the patient's skin to the outer surface of the kidney. The injection needle is then inserted through the guide needle and into the renal capsule. In some configurations, the injection needle can include a 25 G needle having thin walls to minimize the damage to the kidney during needle insertion. The injection needle can include a non-coring (blunt, conical) tip with a side port to minimize tissue damage or clogging of the needle during insertion. The base of the injection needle can include a connector such as, for example, but not limited to, a Luer Lock socket to allow a connection to the transfer tubing. The transfer tubing can include tubing with a connector socket on one end and a plug on the other. The transfer tubing can provide a connection between the injection device and the injection needle. In some configurations, thick-walled microbore tubing with minimal extraneous features can minimize dead volume and compliance in the system.
The vial adaptor can include a fitting that connects to the injection device via a needle-free injection port on one end, and to the injection storage container (vial) via a low gauge needle that pierces the septum on the other end. There is also a retaining feature to hold the vial in place, and a vent filter that prevents vacuum from building up inside the vial. A controller can be configured to adjust the pump rate to reach a pre-selected pressure of the material pumped through the tube and into the injection needle over the course of the start-operation-stop process from run to run, or the pressure can be adjusted to apply a pre-selected pump rate. Monitoring and adjustment of the operations of the device can enable efficient fill of the injection needle, accurate delivery, end stop, and occlusion detection.

The method of the present teachings include the steps of filling, priming, injecting, connecting/disconnecting, monitoring the volume delivered, purging, controlling the user interface, and optionally, recovery of the injection solution. The step of filling can include, after connecting the vial adaptor to the vial and injection device, inverting the vial and driving the injection device plunger in reverse to pull the injection solution into the integrated injection volume container (syringe). The step of priming can include, after performing the filling step, removing the bubbles present in the injection volume container, as well as air in the transfer tubing and injection needle. In some configurations, removing the bubbles can include, but is not limited to including, orienting the injection device so that the tip of the injection volume container is pointing up with the transfer tubing and injection needle attached, and driving the injection device plunger forward until the air is pushed out of the system. The step of injecting can include, after performing the priming step, inserting the injection needle through the guide needle and into the kidney and driving the injection device plunger forward at a controlled rate until stopped by the user, the deposit volume limit is reached, or the injection device software detects a potential issue. The step of disconnecting can include, at various points during the therapy, for example when moving the patient to confirm that the injection needle is positioned correctly, disconnecting the injection needle to transfer tubing Luer Lock union or the transfer tubing to injection device Luer Lock union. During the step of disconnecting, the flow of injection solution from the injection device or transfer tubing (if connected to the injection device) can be interrupted. The step of connecting, after imaging is complete, can include connecting the disconnected Luer Lock union.

The step of monitoring the volume delivered can include, during the injecting step, estimating the amount of volume delivered during the current deposit, and, when the deposit volume limit is reached, automatically interrupting the injection and notifying the user. The step of monitoring can include resetting the deposit accumulator before the next injection, and may be reset at any time the injection device plunger is not in motion to allow for partial volume deposits.

The step of purging, after completing the last full injection action and sensing that there is residual injection solution in the transfer tubing that the healthcare provider wants to inject into the patient, can include disconnecting the injection device from the transfer tubing, performing a fill action with air or saline, reconnecting the injection device, and injecting the air until the transfer tubing is empty. A manual syringe can be used to perform the step of purging if the remaining injection device if power is deemed insufficient to allow fill actions. Controlling injection can include requiring the operator to maintain pressure on the trigger throughout the injection, so that releasing the trigger will immediately stop the injection.

The step of controlling the user interface can include indicating the functions that are currently being performed by the injection device, the status of the battery, and whether any faults or alarms are present. The step of controlling the user interface can also include, during the injection, notifying the user, for example, by haptic feedback, when there is a message that the user can access in order to continue to safely and effectively operate the injection device. The optional step of recovering the injection solution, if, for example, the injection device experience a critical failure, can include removing the injection solution from the injection device and proceeding manually.

Various metrics can be monitored to determine whether the device is accurately providing injections. In some configurations, a mean flow rate accuracy, based on the inner diameter of the syringe, the pitch of the lead screw, the gear train, the motor shaft angular velocity, and the pressure reading. The deposit volume limit of a given injection can be measured by monitoring the total degrees of rotation of the motor shaft as measured by the motor rotary encoder. To detect occlusions, the measured inline pressure can be monitored, the injection can be automatically interrupted, and the device can enter a can begin to shut down when the pressure exceeds a pre-selected threshold. To detect poor or incorrect connections, for example, pressure readings can be monitored during fill and injection actions. If the pressure drop is below a pre-selected threshold, the fill/injection action can be interrupted and the system can return to an idle state. If the motor angular velocity based on the voltage supplied (pulse wave modulation) to the motor and the inline pressure sensor feedback are not within a pre-selected range of each other, for example, because the plunger has hit either the forward or reverse end stop based on the current direction of travel, the motor is stopped and the user is notified. If the power supply charge is below a marginal threshold, the injection device will allow the user to inject the remaining injection solution, but prevent them from performing fill actions. If the power supply charge is determined to be insufficient to safely continue, the injection device can enter the disabled state and ignore commands to move the plunger in the injection direction as well. A power-on self-test (POST) can verify the functionality of, for example, but not limited to, the motor and motor encoder, pressure reading, the display, other user information, and device memory after shipping and storage. A watchdog can be used to monitor for potential runaway software conditions, memory corruption, hardware failures, unanticipated failure modes, or unanticipated user input, and reset the main controller or enter a disabled state or notify the user. The watchdog receives, while the device is operating normally, regular communication from the main controller. Should the software fail to provide this input, the watchdog will reset a master control unit in attempt to fail safe.

### BRIEF DESCRIPTION OF THE DRAWINGS

The present teachings will be more readily understood by reference to the following description, taken with the accompanying drawings, in which:

FIGS. 1A-1B are schematic block diagrams of exemplary systems of the present teachings;

FIGS. 2A-2L are schematic perspective diagrams of exemplary drawings of various configurations of the mechanical system of the present teachings;

FIGS. 3A-3D are schematic perspective diagrams of exemplary drawings of a first configuration of the electromechanical system of the present teachings;

FIGS. 4A-4N are schematic perspective diagrams of exemplary drawings of a second configuration of the electromechanical system of the present teachings;

FIGS. 5A-5B are schematic perspective diagrams of exemplary drawings of a third configuration of the electromechanical system of the present teachings;

FIGS. 6A-6C are schematic perspective diagrams of exemplary drawings of a fourth configuration of the electromechanical system of the present teachings;

FIGS. 7A-7C are schematic perspective diagrams of exemplary drawings of a fifth configuration of the electromechanical system of the present teachings;

FIGS. 8A-8G are schematic perspective diagrams of exemplary drawings of various other configurations of the electromechanical system of the present teachings;

FIGS. 9A-9E are flowcharts of an exemplary method of use of the present teachings; and

FIG. 10 is a schematic block diagram of state transitions of the system of the present teachings.

### DETAILED DESCRIPTION

The device and method of the present teachings are discussed in detail herein. However, various configurations of the device and method of the present teachings are contemplated by the description.

The disposable device for cell delivery to an organ of the present teachings can take several forms, some with manual aspects, some with automated aspects. The organ cells are mixed into a sterile liquid to facilitate injection and optimize the therapeutic benefit in the receiving organ. A liquid containing cells for injection is herein referred to as a medical liquid or a cellular mixture. In one exemplary treatment, organ cells are stored in DMSO. The DMSO/organ cell mixture or medical liquid is stored in a frozen condition and then thawed before being injected into a body. Exemplary configurations of the injector of the present teachings include, but aren't limited to including, a trigger mechanism, a syringe, and a pressure sensor. Exemplary configurations include any of a sealed enclosure, an integrated syringe with volumetric markings, a pressure sensor that directly mates to the syringe, a motor and gearbox used to drive a lead screw that moves the syringe plunger through a syringe, and other features. In an aspect, the device provides volume and rate measured injection of cellular mixture into an organ. In an aspect, the sealed enclosure protects the functional mechanisms from debris/liquid ingress and holds components in place relative to each other The cellular mixture can be drawn into or pulled into the device, or can be provided to the device by a pre-filled syringe. In an aspect, a force, possibly provided by a mechanical spring or by an automated mechanism, drives the lead screw to force the syringe plunger into the syringe, and move the cellular mixture out of the syringe. Data from a pressure sensor can be used to control liquid flow to a pre-selected pressure. In an aspect, in case of failure to deliver the cellular mixture, the cellular mixture can be extracted from the syringe to a holding container for storage and use in another procedure.

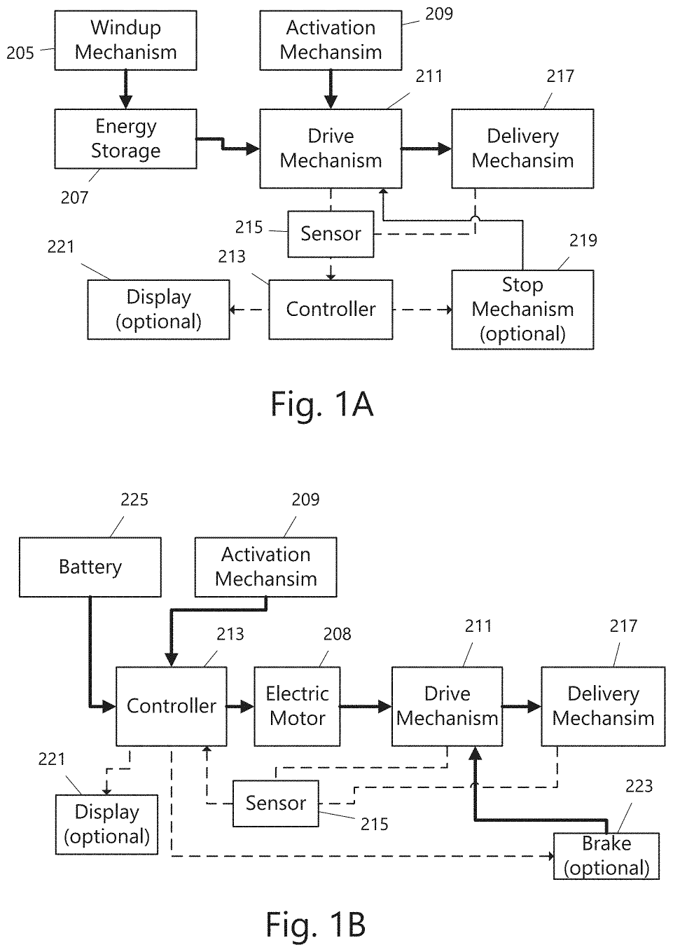

Referring now to FIGS. 1A-1B, various exemplary configurations of the system of the present teachings are shown in schematic block diagram form. In an aspect, the delivery device (FIG. 1A) of the present teachings includes windup mechanism 205 that charges the energy storage mechanism 207 to provide force to the drive mechanism 211. The drive mechanism 211 engages with delivery mechanism 217 to deliver the cellular mixture to the organ when activation mechanism 209 is depressed or otherwise caused to communicate with drive mechanism. In an aspect, sensor data from sensor(s) 215 is processed by controller 213 to adjust the characteristics of the cell delivery. In an aspect, characteristics can include, but are not limited to including, pressure. In an aspect, activation mechanism 209 includes a trigger that releases drive mechanism 211. For example, activation mechanism 209 includes a device that rests in engagement with a drive gear, and is moved to release the drive gear, setting off a chain of movement causing drive mechanism 211 to apply force to, for example, a lead screw. The lead screw eventually engages with delivery mechanism 217, for example, a syringe plunger that delivers the cellular mixture to the organ through a syringe. In an aspect, windup mechanism 205 actuates a spring such as a torsion spring positioned in a timer gear train, as well as a motor mechanism such as storage and torque drums. Releasing the gear train engaged with the drive gear activates motor mechanism and the timer which controls the release of energy by motor mechanism 207.

Optionally, a delivery stop mechanism 219 may be included that freezes drive mechanism 211 in position. Such an action ensures that no further transfer of cellular mixture will happen, no matter the status of activation device. Additionally, the delivery device may optionally include a display 221 may be built into the device. In an aspect, the display 221 indicates, for example, but not limited to, the status of the device, the volume of cellular mixture left to be delivered, error messages, and the delivery pressure. In an aspect, the display includes a code. In an aspect, the code is displayed pictorially. In an aspect, the code is depicted in color.

Referring now to FIG. 1B, in an aspect, an electromechanical configuration of the present teachings includes battery 225 and electric motor 208 that provides force through drive mechanism 211 to delivery mechanism 217. In an aspect, energy can be stored in battery 225 including but not limited to the following battery types: dry cell, lithium-ion, zinc-carbon, alkaline, lithium-iron sulfide, nickel-metal hydride, and nickel-cadmium In an aspect, battery 225 is replaced by other forms of electrical energy storage such as, for example, but not limited to, or capacitors. Motor 208 is controlled by electromechanical controller 214. Electromechanical (EM) controller 214 receives and processes sensor data from sensor(s) 215. In an aspect, sensor data includes pressure data indicating the pressure of the cellular mixture as they are delivered to the organ. Other data can be gathered by sensors associated with the device. In an aspect, cell characteristic sensors provide data to controller 214. In an aspect, sensor data are used by Controller 214 to adjust the volume of cellular mixture delivered and the pressure at which the mixture is delivered. Other types of control are contemplated by the present teachings. In an aspect, Controller 214 associates collected sensor data with desired actions through an algorithm, a recipe, or a dynamically-varying association, and directs action based upon the results of the application of the method to the data. Commands are generated by Controller 214 to the drive mechanism 211 based on the directed actions. Drive mechanism 211 provides mechanical force to the delivery mechanism 217 causing the cellular mixture to be delivered to the organ.

Continuing to refer to FIG. 1B, the delivery device may include a brake 223. Brake 223 inhibits further movement of drive mechanism 211 regardless of the status of activation mechanism 209. In an aspect, brake 223 includes a mechanical stopper that physically inhibits movement. In an aspect, brake 223 includes electrical signals, possibly augmented by mechanical means, that stop the motion of drive mechanism 211 or the electric motor. Other possible brake configurations are contemplated by the present teachings including a signal to the controller 213 to stop the electric motor 208. Additionally, the delivery device may include a display(s) 221. In an aspect, display(s) 221 can be used as described herein to inform the user of various characteristics of the device, the cells, and the environment of the cells.

### Mechanical Injector

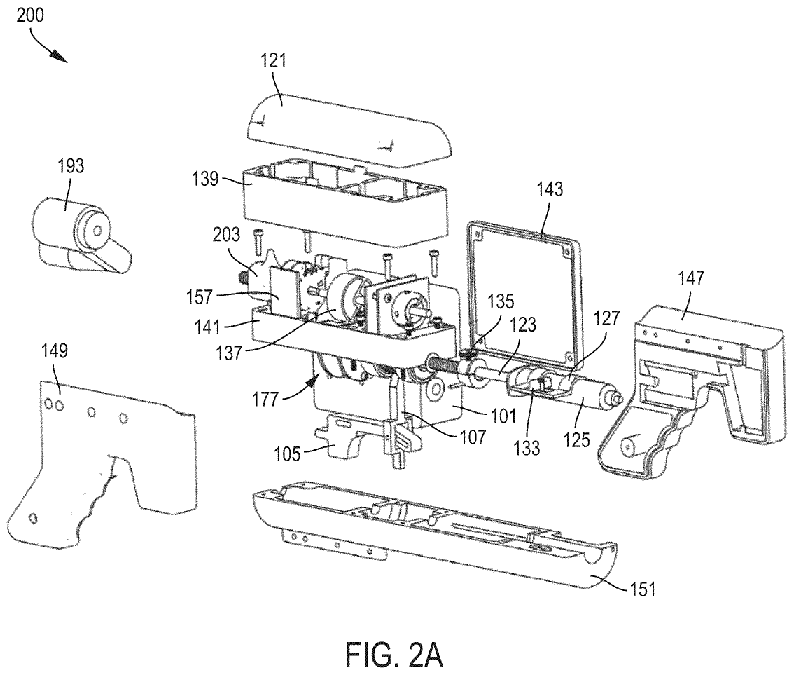

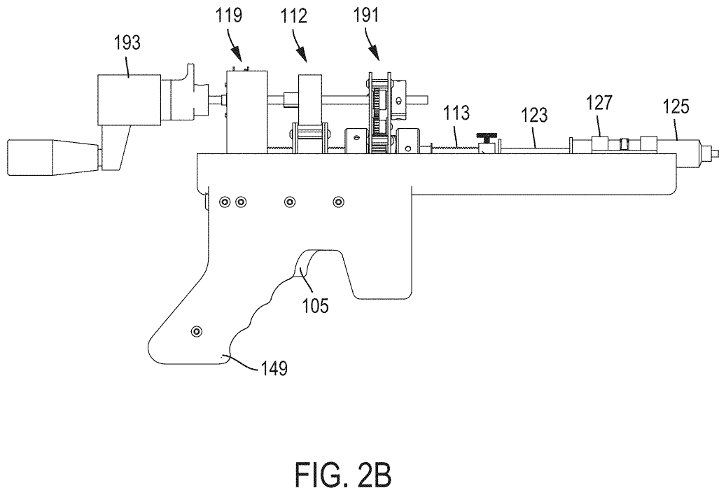

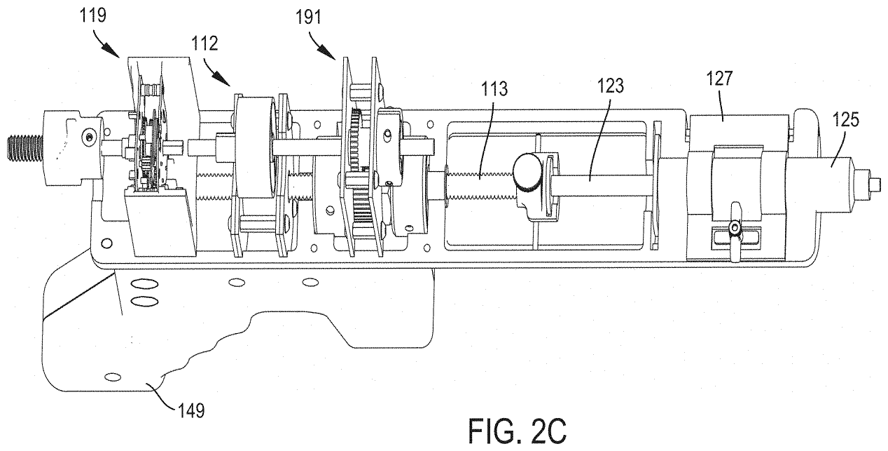

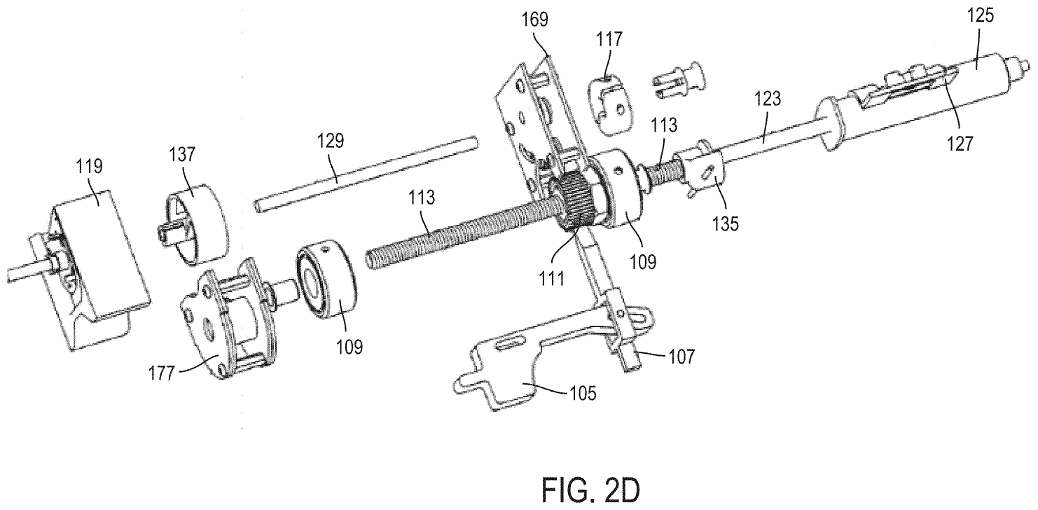

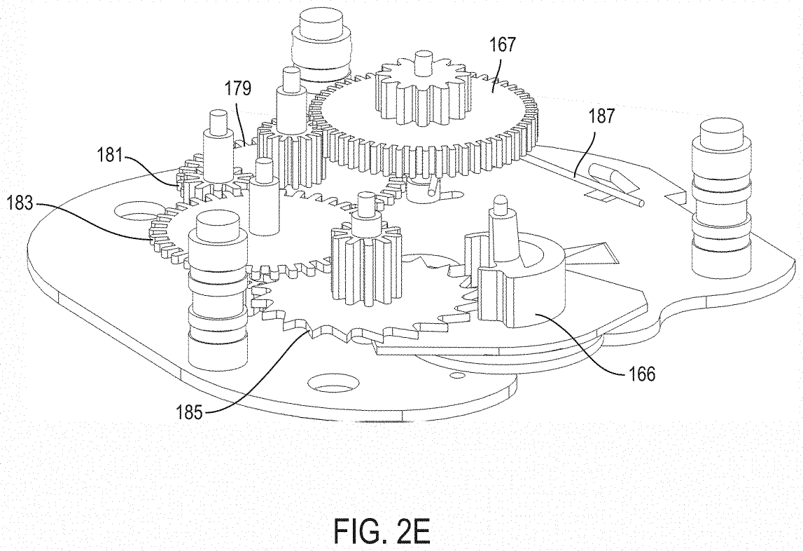

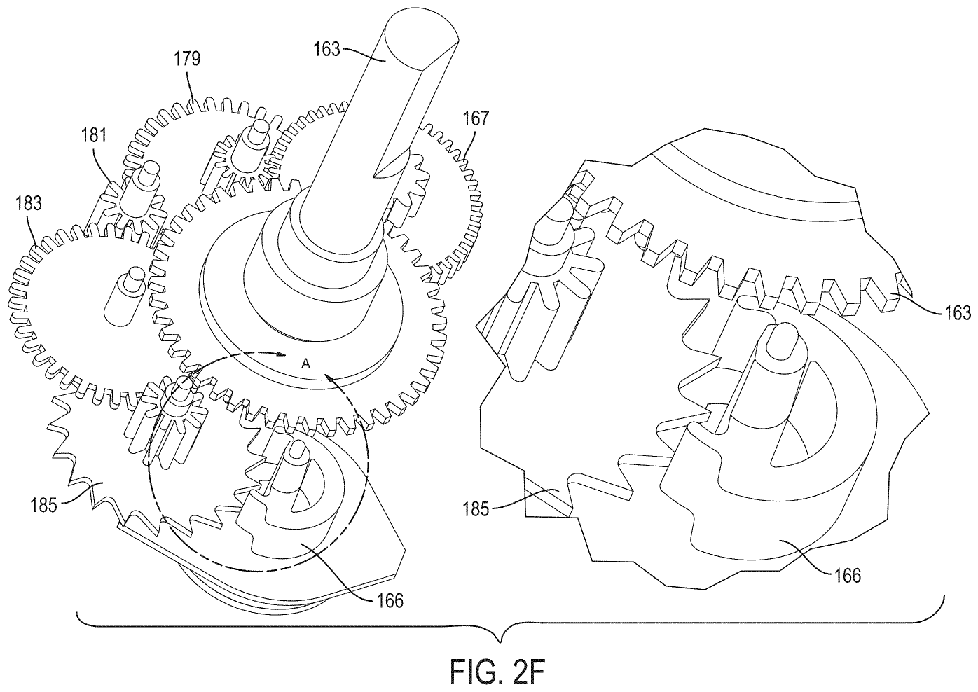

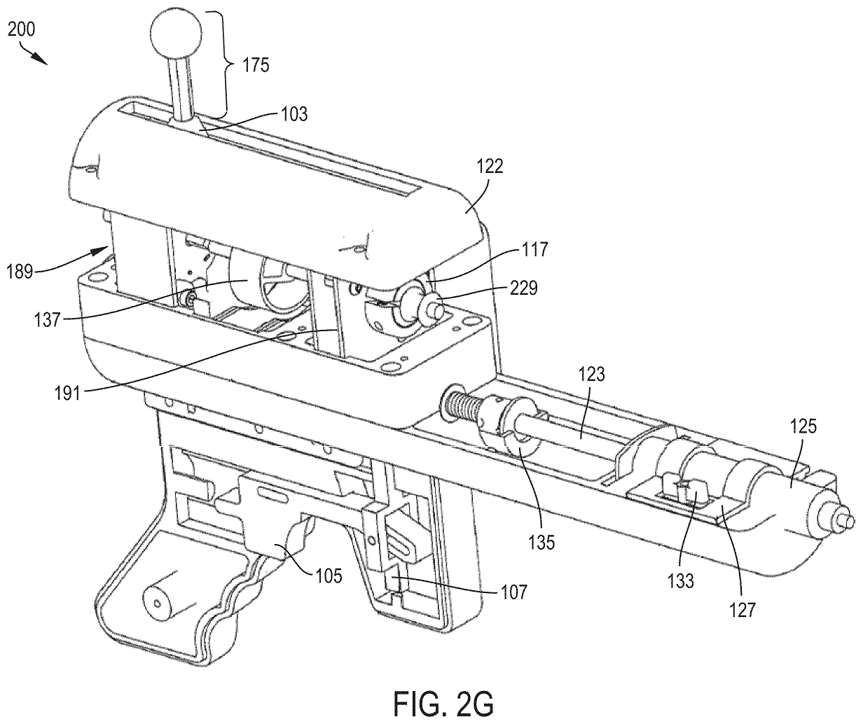

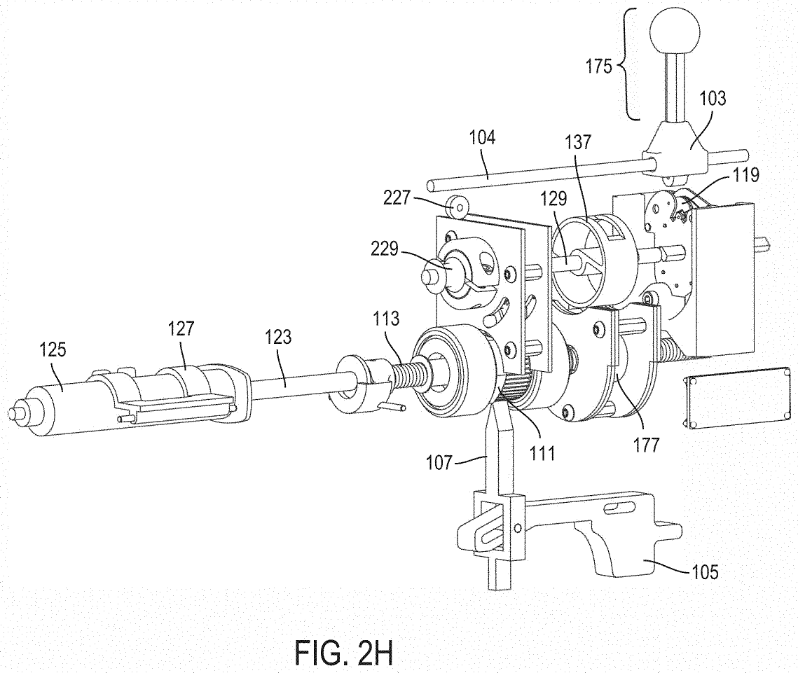

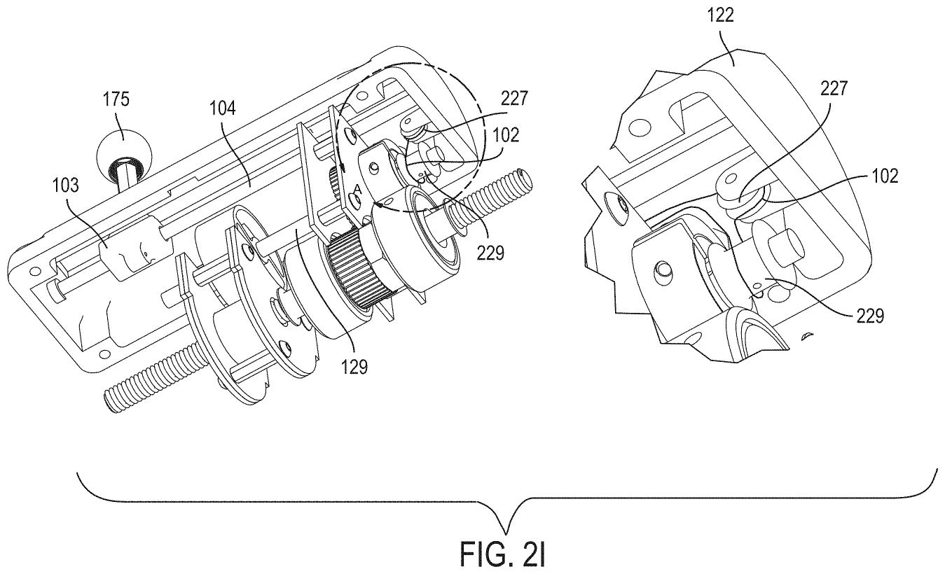

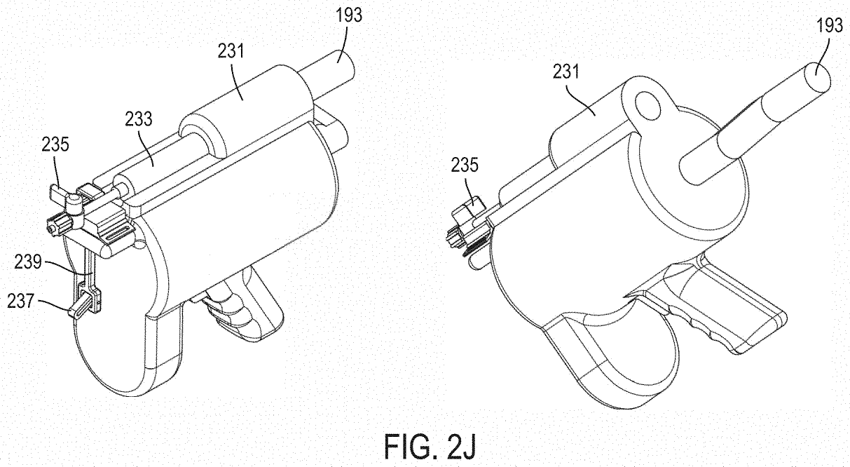

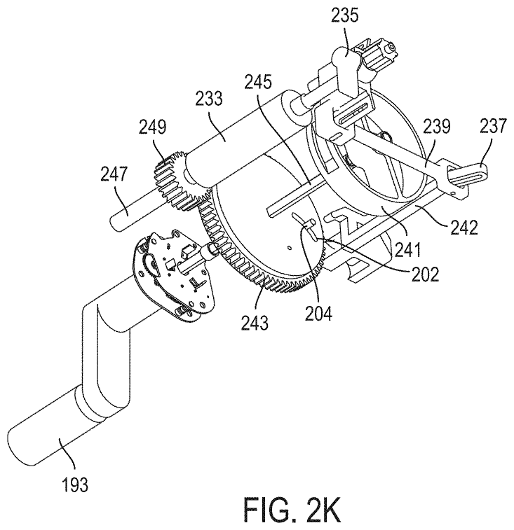

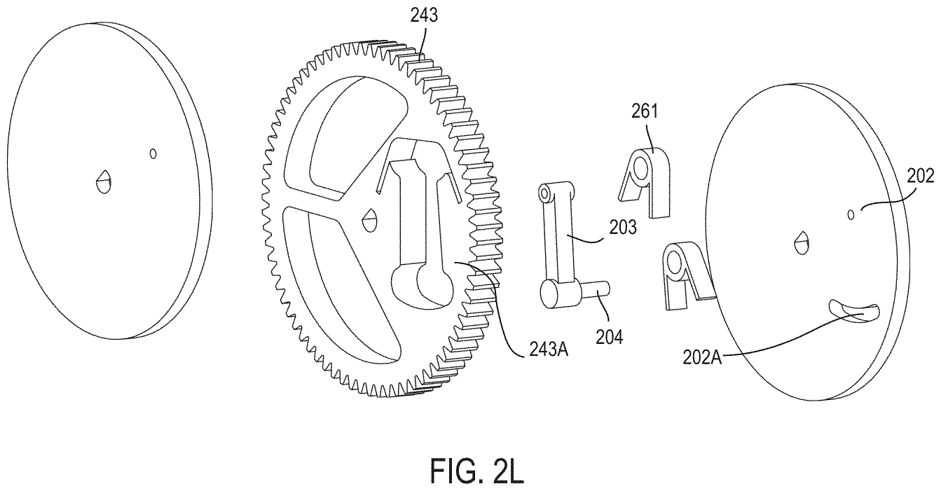

Referring now to FIG. 2A, first exemplary configuration 100 (FIG. 2A) of a mechanical cell injector of the present teachings is shown. Disposable mechanical system 100 (FIG. 2A) includes a protective casing, a delivery mechanism, a trigger, and an activation mechanism moving the cells that are in the delivery mechanism into a receiving organ. The protective casing can be formed in multiple interconnecting sections or can be formed as a single section. In an aspect, the protective casing includes body lid 121 (FIG. 2A), body upper 139 (FIG. 2A), mid body 141 (FIG. 2A), and handle casing(s). In an aspect, handle casings include right side handle casing 149 (FIG. 2A) and left side handle casing 147 (FIG. 2D). Other casing configurations are contemplated by the present teachings. Operationally, the device is spring-wound with a timing escapement that drives a syringe plunger at a fixed rate. The device has a trigger that controls when the spring is released to drive the plunger. A pressure sensor is installed in-line with the nozzle of the syringe to detect pre-selected changes in pressure at the needle tip. The benefit of this device is that it is human driven. Delivery energy input into the device is done by the operator, but unlike depressing the syringe by hand, the device drives delivery of liquid in a syringe at a fixed rate across a range of viscosities. The delivery system is purely mechanical and rechargeable by winding the spring mechanism. In an aspect, a syringe plunger is pushed by a lead screw. In an aspect, component 135 (FIG. 2A) attaches component 136 (FIG. 2A) to the end of lead screw 113 (FIG. 2A) that interfaces with the syringe plunger. In FIGS. 2J-2L, the syringe plunger is replaced by a plunger seal attached to the end of a lead screw. An axially stable drive nut drives the lead screw into the syringe plunger by turning in place. The lead screw is held rotationally stable so that it translates forward and does not rotate with the nut. The drive nut has gear teeth around its exterior and is connected to a drive shaft via a short gear train. The drive shaft has a torque drum around which is wound a spring. The selected spring is designed to have multiple turns with constant torque output from each turn. The gear train that connects the drive shaft to the drive nut can be geared up or down to meet a pre-selected number of drive nut turns per volume of the stroke at the syringe. This is a function of the syringe geometry and the pitch of the lead screw. The drive shaft is also connected to a mechanical timing mechanism (akin to a spring-wound egg timer). This mechanism ensures the drive shaft rotates at a fixed rate, limiting the rate of delivery. The rate of this timing mechanism could either be static, or could be shifted between multiple fixed rates by means of a drive train with a “gear shift” that alters which gears connect to the drive shaft. In an aspect, the trigger is a spring-loaded mechanism that, at rest, pushes a wedge into the teeth of the drive nut, preventing its rotation and thus blocking delivery of the medication. In an aspect, the start/stop mechanism includes a friction break. In an aspect, the start/stop mechanism includes an axial mechanism. Upon user pulling the trigger, the wedge is pulled out of the path of the drive nut teeth, and the device is freed to continue delivery. This safety mechanism allows the operator to release the trigger in an emergency and the springs will push the wedge back into the gear teeth making the device stop delivery. Releasing the trigger does not unwind the spring, and pulling the trigger resumes motion. The spring is must be rewound after a pre-selected number of aliquots has been delivered. There are several options to achieve this, such including a crank or knob on the rear of the drive shaft, a ratcheting lever on the side of the device, or a linear slide with a gear rack that engage with a bevel gear on the drive shaft. In an aspect, when the operator winds the spring, the drive nut is not spun in the reverse direction as this would retract the syringe plunger. An intermediate gear in the gear train is therefore introduced that is pushed out of contact with the drive nut during the wind up action, thereby breaking the gear train. This gear is pushed back into contact with the drive nut when the spring is unwinding. A pressure sensor is attached to the luer lock connector at the tip of the syringe. It is connected to a microprocessor that is stored in the electrical box on the side of the device. A rocker switch connects battery power to the microcontroller and pressure sensor. The microcontroller monitors the pressure at the sensor and stores a rolling buffer of pressure from the last 3 seconds, sampling at 10 Hz. The processor compares new measurements with the measurement from 3 seconds previously, and looks for drops over that interval (e.g. >50 mmHg) that might indicate the needle tip moving into a blood vessel or other non-intended tissue area. The drop threshold and time-frame can be variably adjusted. An LED on the top of the electrical box could, for example, change from green to red to indicate to the user that there has been such a drop in pressure. The user may release the trigger upon seeing the red light, then adjust the location of the insertion needle before restarting injection of the aliquot by pulling the trigger again.

Referring now to FIGS. 2B-2C, within the environmental case, the delivery mechanism and the major parts of the activation mechanism of an exemplary configuration are shown. In an aspect, the delivery mechanism includes syringe 125 coupled with plunger 123. In an aspect, syringe 125 is held in place by syringe restraint 127, securing syringe 125 against movement when under pressure by lead screw 113. Other delivery mechanisms are contemplated by the present teachings. The activation mechanism provides a desired amount of pressure to force the cells from the delivery mechanism into the organ. The amount of pressure applied is based at least on avoiding cell hemolysis, the delivery mechanism, and the resistance of the organ, possibly affected by the environment surrounding the organ. In an aspect, applied pressure is automatically monitored to enable adjustment of the applied pressure. In an aspect, a pressure sensor/needle/administration kit can be attached to the device, and the pressure sensor can be enabled. A rocker switch, among other types of switches, can be used to enable the sensor. Referring to FIGS. 2B-2C and 2D, in an aspect, the delivery device includes timing mechanism 119, torque drum assembly 112, and drive nut assembly 191. In an aspect, use of the device of the present teachings is begun by charging the torque drum assembly 112, which also called a torsion spring or main spring mechanism. With respect to charging, in an aspect, torque drum assembly 112 includes torque drum 137 (FIG. 2D), storage drum 177 (FIG. 2D) and a torsion spring (not shown). In an aspect, storage drum 177 (FIG. 2D) and torque drum 137 (FIG. 2D) are arranged such that a strip of spring sheet metal has two ends, ends being attached to one of the drums. The strip of spring sheet metal is coiled on storage drum 177 (FIG. 2D) in a relaxed state. The motor mechanism is chargeable by rotating torque drum 137 (FIG. 2D) thereby coiling the strip of spring sheet metal onto torque drum 137 (FIG. 2D) and bending the torsion spring or strip of spring sheet metal the other way round than in the relaxed state thus arriving in a charged state with the strip of spring sheet metal tending to re-coil onto storage drum 177 (FIG. 2D) thereby generating a torque. In an aspect, torque generation methods that result in constant torque and thereby enable constant delivery speed and force, are selected. When charging is complete, the device can be used operationally.

Referring to FIGS. 2D, to use the device operationally, trigger 105, when at rest, slides a wedge between the teeth of drive gear 111, preventing its rotation and thus blocking delivery of the cells within syringe 125. Upon user pull trigger 105, wedge 107 is pulled out of the path of the teeth of drive gear 111, and the device is freed to continue delivery. In an aspect, if trigger 105 is spring-loaded, the operator can “drop” or release the trigger 105 in an emergency and the springs push wedge 107 back into the gear teeth making the device stop delivery. In this manner, the trigger 105 is released and re-depressed after aliquots. This can prevent unintentional over-delivery of medication. It also bestows upon the device a sort of memory; if trigger 105 is dropped part-way through an aliquot, then trigger 105 is re-depressed, the device will complete the remained of that aliquot instead of starting a new one. In an aspect, a pressure sensor (not shown) can be attached to the connector at the tip of the syringe to detect pressure in syringe 125 (FIG. 2B). In an aspect, the pressure sensor is connected to a processor that monitors the pressure at the sensor. In an aspect, the processor stores a rolling buffer of pressure from a pre-selected amount of time, sampling at a pre-selected rate. The processor compares new measurements with the measurement from the stored rolling buffer, and looks for drops over the pre-selected interval (e.g. >50 mmHg) that might indicate the needle tip is moving into a blood vessel or other non-intended tissue area. In an aspect, the drop threshold and time-frame can be adjusted. In an aspect, the device is single-use. In an aspect, the device, or parts of the device, are durable.

Referring now to FIGS. 2C, timing mechanism 119 can moderate the release of energy stored in torque drum assembly 112. When gear nut assembly 191 is released to move, torque shaft 129 (FIG. 2E) is free to move as well, allowing timing mechanism 119 to begin a moderated release of energy from torque drum assembly 112 and the rotation of the gear train in gear drive assembly 191, thus driving lead screw 113 which pushes plunger 123 into syringe 125.

Referring now to FIGS. 2E, 2F the timing mechanism 119 (FIG. 2E) includes timer gear mount 159, and timer shaft 163 that is allowed to rotate by the release of the torsion spring in the torque drum assembly 112 (FIG. 2E), which allows timer sprung gear 167 to rotate. Timer sprung gear 167 also rotates timer gears 179/181/183/185. Timer gear 185 interfaces with escapement 166 that periodically releases the gear train to move, thus metering the flow of cellular mixture into the organ.

Referring now to FIGS. 2G-2I, in another exemplary configuration, an activation mechanism includes a movable device that charges the device. When ball knob 175 (FIG. 2H) slides along bolt rail 104 (FIG. 2H) under body lid 122 (FIG. 2I), primary pulley 227 (FIG. 2I), secondary pulley 229 (FIG. 2I), pulley belt 102 (FIG. 2I), and bolt pivot 103 (FIG. 2H) operate cooperatively to send a rotational force to torque shaft 129 (FIG. 2H). The force causes torque drum 137 (FIG. 2H) to rotate cooperatively with storage drum 177 (FIG. 2H) to charge torque drum mechanism 112 (FIG. 2C). Other charging mechanisms are contemplated by the present teachings.

Referring now to FIGS. 2J-2, in yet another exemplary configuration, a syringe stopper is attached to the end of the lead screw, removing the syringe plunger from the design, saving length. Axially stable drive nut 249 (FIG. 2K) drives lead screw/plunger 247 (FIG. 2K) into syringe 233 (FIG. 2K) by turning in place. Lead screw/plunger 247 (FIG. 2K) is held rotationally stable so that it translates forward and does not rotate with drive nut 249 (FIG. 2K). Drive nut 249 (FIG. 2K) has gear teeth around its exterior that mesh directly with drive gear 243 (FIG. 2K). Drive shaft 245 (FIG. 2K) has torque drum 241 (FIG. 2K) around which is wound a spring (not shown). Housing 231 (FIG. 2O) allows for a relatively large spring, allowing a relatively large torque. The selected spring is designed to have multiple turns with constant torque output from the turns. Drive gear 243 (FIG. 2K) includes springs 261 (FIG. 2L) that are sized such that one full rotation of drive gear 243 (FIG. 2K) results in one full aliquot of liquid delivery. In an aspect, one full rotation of the drive gear equals one aliquot, and the device includes a feature that forces the operator to release the trigger after each aliquot before the beginning the next aliquot as a safety mechanism to prevent over-delivery. Timer assembly 251 (FIG. 2K) ensures drive shaft 245 (FIG. 2K) rotates at a fixed rate, limiting the rate of delivery of the cells.

Continuing to refer to FIGS. 2J-2L, in an aspect, the device includes stopcock 235 (FIG. 2K) that can ensure that the cells are delivered at a pre-selected rate, and that the cell aliquot is delivered in full in sync with the mechanical delivery. Stopcock 235 (FIG. 2K) is operably coupled with stopcock pivot 239 (FIG. 2K) which engages with trigger 237 (FIG. 2K) to activate cell delivery. In an aspect, force is applied to lead screw/plunger 247 (FIG. 2K), for example, by rotating crank 193 (FIG. 2O). Drive shaft 245 (FIG. 2K) can be rotated a number of times to move lead screw/plunger 247 (FIG. 2K) a desired amount, for example, four times. Delivery of the cells begins when trigger 237 (FIG. 2K) is depressed and drive nut 249 (FIG. 2K) is released. Drive shaft 245 (FIG. 2K) is rewound at pre-selected intervals. When present, a display (not shown) can warn the user if the pressure drops by a pre-selected amount over a pre-selected timeframe, for example, but not limited to, 50 mmHg over 3 seconds.

### Limiting Delivery of Liquid Per Trigger Pull

In an aspect of the device includes a mechanism that limits the amount of cellular mixture delivered per pull of the trigger. The delivery limit mechanism allows the user to delivered a quantified volume of cellular mixture to a given site. Continuing to refer to FIGS. 2J-2L, while trigger 237 (FIG. 2K) is depressed, an activation mechanism including slanted beam 242 (FIG. 2K), is brought into close proximity to the front face of drive gear 243 (FIG. 2K). Within the activation mechanism is a rotating arm 203 that is held at the center of a swing arc 202A (FIG. 2K) by preloaded torsion springs 261 (FIG. 2L). The end of the arm 203 includes tooth 204 (FIG. 2K) that protrudes out of the front face of gear 243 (FIG. 2K). When an aliquot is completed, tooth 204 (FIG. 2K) comes into contact with slanted beam 242 (FIG. 2K) of the trigger mechanism. The slanted nature of the beam pushes the arm rotationally, causing one of torsion springs 261 (FIG. 2L) to compress further. The tooth 204 swings to the end of travel slot 202A (FIG. 2K) while the arm swings to the surface of cutout 243A. The slanted bean 242 stops the rotation of the pin 204 and thus stops the rotation of drive gear 243 (FIG. 2K), and thus stops delivery of liquid. When the trigger is released, slanted beam 242 (FIG. 2K) is moved out of the path of tooth 204 (FIG. 2K), which allows the drive gear 243 to rotate and restart delivery of liquid. Once the pin 204 is no longer blocked by the slanted beam 242, the arm 203 returns to its neutral position due to the two torsion springs 261 balancing their forces applied to the arm 203. In the neutral position of the arm, the tooth 204 (FIG. 2K) may be in the center of slot 202A and rotationally passed the slanted beam 242 (FIG. 2K), so that when trigger 237 (FIG. 2K) is re-depressed, the beam is no longer blocking tooth 204 (FIG. 2K) until the drive gear 243 completes another rotation and in some instances delivers another aliquot.

This approach can be expanded beyond one rotation of the drive gear per trigger pull. In another embodiment, there are 2, 3, or more assemblies of arms 203 each with a tooth 204 and centering springs 261. The drive gear 243 and liquid delivery will stop as each tooth 204 is engaged by the slanted beam 242. The drive gear and liquid delivery will then restart when the trigger is released, allowing the arm 203 to a neutral position and then re-pulled. In this way, smaller aliquots of liquid could be dispensed per rotation of the drive gear 243 by adding additional arms 203 to the drive gear 243. The mechanism including parts 202, 203, 204, 242, 243 and 261, described herein and referring to FIGS. K and L may also be applied to any of the exemplary configurations shown in FIGS. 2A-2J.

Continuing to refer to FIGS. 2K-2L, the arm 203 can also swing in the other direction. This, combined with the fact that the spring can store enough energy for a syringe worth of aliquots, means that the device can support vial decanting. To achieve this, trigger 237 (FIG. 2K) is held depressed and crank 193 (FIG. 2K) is rotated to cause torque shaft 245 (FIG. 2K) to rotate in the opposite direction with respect to the direction during an aliquot delivery for a pre-selected number of full winds. As the drive gear rotates in the opposite direction, tooth 204 (FIG. 2K) hits the obliquely slanted side of slanted beam 242 (FIG. 2K), which pushes the tooth upwards and over the top of beam 242 (FIG. 2K) resulting in resetting the arm back into its neutral position by torsion springs 261 (FIG. 2L). The rotation of drive gear 243 (FIG. 2K) spins drive nut 249 (FIG. 2K) in reverse, pulling the stopper backwards and drawing a vacuum in the syringe, allowing it to pull medication into it. In addition, the reverse rotation of the drive gear winds the torsion spring back on the torsion drum 239. Because energy is being cranked into the device during vial decanting, crank 193 (FIG. 2K) can be removed, and no further energy needs to be input into the device during the remainder of the medication delivery process.

### Electro-Mechanical Injector

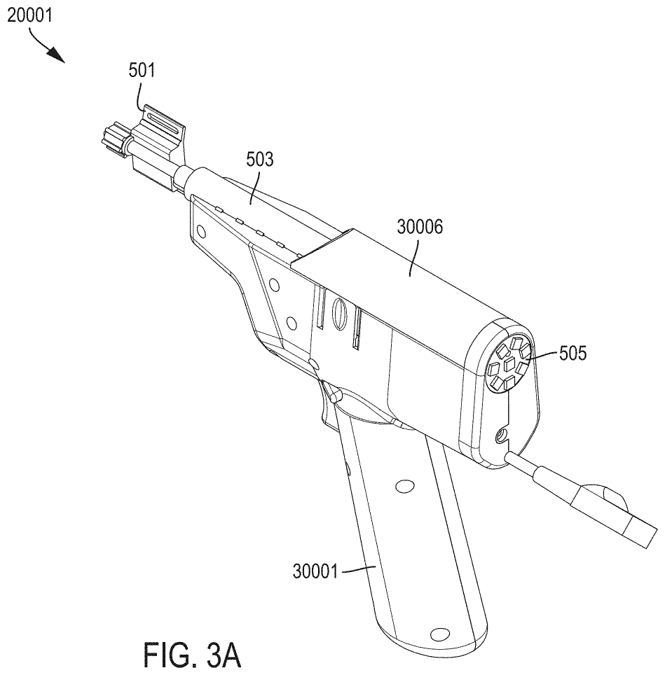

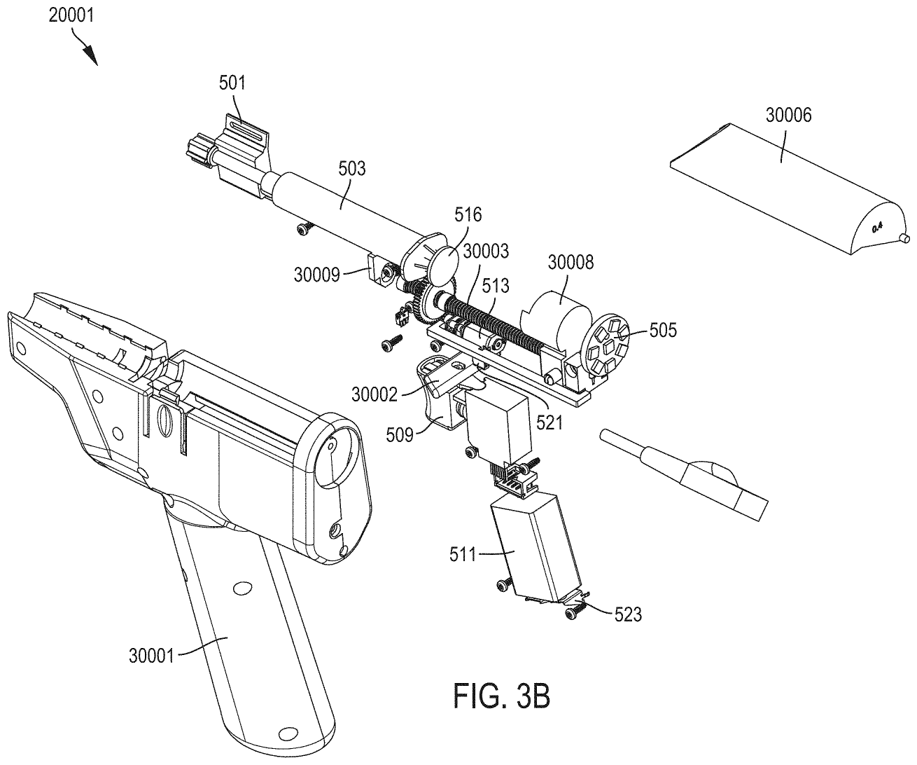

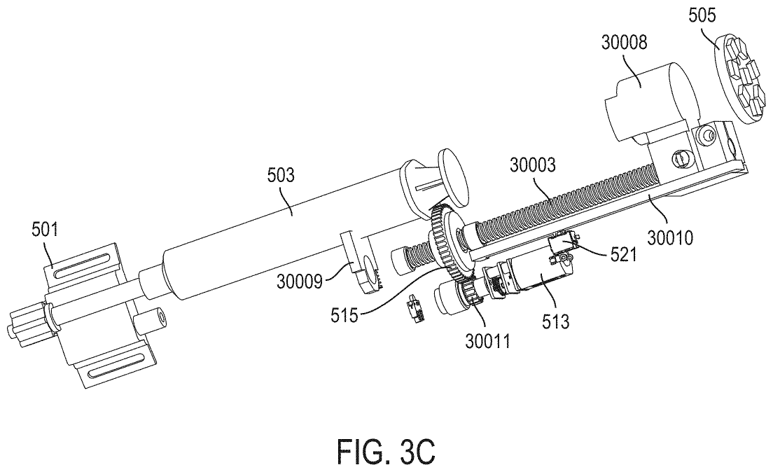

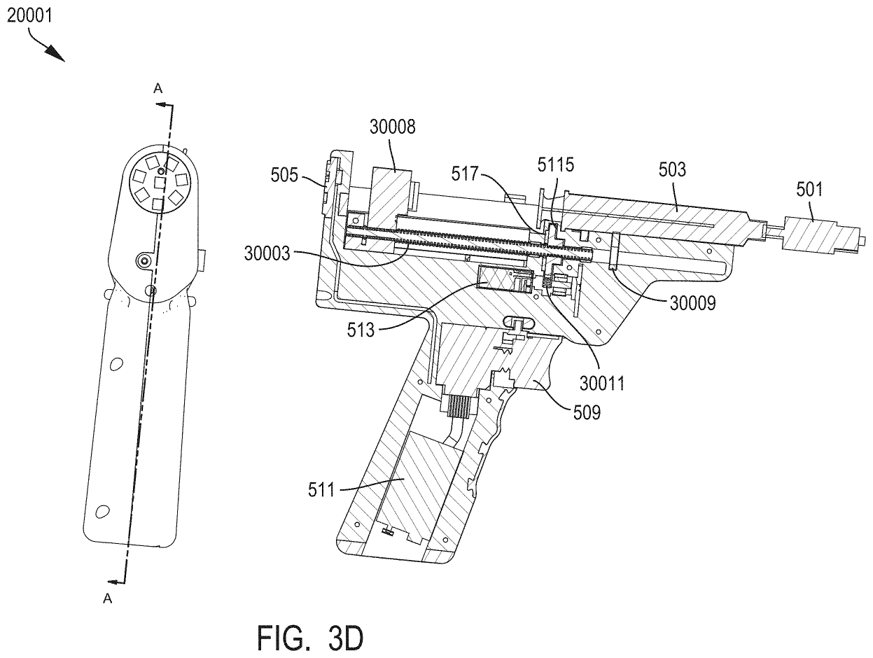

Referring now to FIGS. 3A-3D, a first exemplary configuration of the electromechanical injection device of the present teachings is shown. The disposable device includes an environmental casing, power, a delivery mechanism, an activation mechanism, a motor mechanism, and optional status display(s) and a charging option. The charging option can be used in devices with field-installed syringes, and the electromechanical actuator portion of the device is durable and rechargeable or contains swappable batteries. Referring to FIG. 3A, exemplary device 20001 includes syringe cover 30006. In an aspect, syringe cover 30006 is hinged to device case 30001. In an aspect, syringe cover 30006 is removable/replaceable. In an aspect, syringe cover 30006 is shaped to allow placement of syringe 503 in case 30001. In an aspect, the syringe cover 30006 may latch to the device case 30001, thereby securing the syringe in the case. In an aspect, components of exemplary device 20001 are protected from environmental contaminants by case 30001. In an aspect, case 30001 securely protects internal components of device 20001 from liquid contaminants. In an aspect, case 30001 securely protects components of device 20001 from micro-contaminants. In an aspect, case 30001 securely protects components of device 20001 from macro-contaminants. In an aspect, case 30001 includes internal features that hold the components in place as device 20001 is used.

Referring now to FIG. 3A, depicted is an electromechanical configuration of the injection device. Use of this device can include steps of loading the empty syringe into the device and closing the lid. In some configurations, an indication to the user that the syringe is properly loaded can be provided. For example, an LED can switch from blue to green when the syringe is properly loaded. A direction selector can be toggled to reverse the device.

### LED Light Signals

In some configurations, an LED array can display a reverse pattern. To extract liquid from a vial, the trigger can be depressed and released when the liquid is extracted. In some configurations, the LED array can display a moving pattern, for example, while the trigger is depressed. The vial can be replaced with a pressure sensor and needle set, and the cable for the pressure sensor can be connected to the device. The direction selector can again be toggled to place device in dispense mode. In some configurations, the LED array can display a forward pattern. Next, the trigger is depressed and held to initiate dispensing of the liquid. At this time, the motor will spin up to reach the proper pressure to dispense the liquid, and then decelerate to delivery speed. In some configurations, the LED array can display a moving forward pattern. The motor can automatically be stopped when a pre-selected condition occurs. For example, the motor can be stopped when a pre-selected amount of liquid has been dispensed. This situation can be indicated by the pattern on the LED array. The motor can automatically be stopped when a pressure drop has been detected. In some configurations, the LED array can flash a pre-selected color, and a release of the trigger can clear the fault. The motor can automatically be stopped when the trigger is released. At this time, the motor can retract the plunger to equalize pressure and prevent further dispensation of the liquid. Manual retraction of the plunger is also possible to actuate a manual recovery mechanism.

Continuing to refer to FIG. 3A, the pressure sensor 501 senses the pressure in the syringe, enabling, among other things, the user to observe if the tip of the needle or any part of the liquid pathway is occluded. The syringe can be held in a device case 30001 with a bayonet assembly. Alternatively or in addition, the syringe cover 30006 may latch to the device case 30001, thereby securing the syringe in the device case 30001. The device cover 30001 does not necessarily seal to the syringe. When the lead screw is being driven forward, the gear is rotating around the lead screw. Continuing to refer to FIG. 3A, there are multiple ways that the user can interact with the device. The direction that the plunger moves can be selected, and the trigger can be depressed. In some configurations, a photo interrupter can indicate the status of the trigger. As the trigger is pulled back, it comes into contact with a steep face, and a tactile click actuates the trigger. Changing directions can trigger tactile feedback to the user. The system can be sealed. A tunnel can be formed from one side of the device to the other to protect the printed circuit board (PCB) from moisture. The device can include a power supply button to interact with a terminal of the power supply. In some configurations, the button can include a plastic tab between the power supply terminal and the spring contact. When the user presses on the dome formed by the button, the button inverts and allows the spring contact to interact with the power supply terminal.

Continuing to refer to FIG. 3B, the motor can have multiple speeds. A relatively fast speed can be used to accomplish a quick prime of the injection needle. During rapid fill, the liquid can be pumped at a rate of, for example, but not limited to, 5-6 ml/min. Back pressure from the cells can build up at the intersection of the tube and the injection needle. The processor can slow the motor when the pressure, as detected by the pressure sensor, reaches a pressure threshold which can be in the range of 300-1000 mmHg. In an aspect the pressure threshold is 800 mgHg. The motor 5013 can drive the pump at its relatively slower speed in order to deliver the liquid at a speed that is consistent with the uptake rate of the organ. During operation, the liquid can be pumped at a rate of, for example, but not limited to, 1 ml/min. The pressure can be monitored during operation to check on delivery speed. The user can stop operation by releasing the trigger 509. The orientation of the switch 30002 can direct the processor to run the motor in reverse. The pump may stop when the pressure measured by the pressure sensor 501 drops below a second threshold such as less than 40 mmHg, or when the leadscrew 30003 reaches a limit or the user releases the trigger. During operation, if the power supply voltage drops below a pre-selected threshold, the processor can raise an alert. The user can command the remaining aliquot to be delivered with the trigger 509. When the plunger reaches the end of stroke in the syringe barrel, The processor can detect when the plunger reaches the end of stroke in the syringe barrel by comparing the change in pressure at sensor 501 and the rotational velocity of the motor 513. An end of stroke may be declared if the motor velocity is inconsistent with the measure pressure. Two examples, but not the only examples, of end of stroke inconsistencies are the motor shaft is not rotating, or the measured pressure is falling, but the rotor is still rotating. The controller will put the pump in an idle state until the user changes an input. Until the user changes direction, the microprocessor will reject any attempt to move forward. Raising alarms can include providing haptic feedback such as, but not limited to, vibration.

Referring now to FIGS. 3B-3D, in an aspect, components of device 20001 include battery 511, powering motor 513. In an aspect, battery 511 is sized to provide the desired power while allowing for a relatively small footprint of device 20001. In an aspect, battery 511 is spring mounted in case 30001 with leaf spring battery contacts 523. In an aspect, power is provided by light and/or movement energy conversion devices. In an aspect, power is provided by a rechargeable battery. In an aspect, power is provided by the electricity grid. Trigger 509 activates motor 513 which rotates gear 30011 (FIG. 3C) and gear 515. In an aspect, trigger 509, when depressed, signals the controller to spin the motor in a direction and at a speed based in part on the pressure measured by sensor 501 and syringe position. The controller provides electrical power transmission from battery 511 to motor 513. In an aspect, direction switch 30002 establishes the direction that lead screw 30003 is driven. At a first orientation of direction switch 30002, lead screw 30003 is driven in a first direction, and at a second orientation of direction switch 30002, lead screw 30003 is driven in a second direction that is opposite to the first direction. When lead screw 30003 is driven in a first direction, for example, counterclockwise, coupler 30008, which is operably coupled with syringe plunger 516, moves syringe plunger 516 into syringe 503, thus displacing the contents in syringe 503. Gear 515 rotates when pinion 30011 is driven by the motor. Gear 515 has matching ACME thread in the bore, and pulls lead screw 30003 through it, which also pulls coupler 30008 to actuate the syringe plunger. Coupler 30008 is fixed attached to the lead screw 30003 and prevents the lead screw from rotating about its longitudinal axis. Syringe presence detection switch actuator 30009 includes a clearance pocket or opening 30009A to accommodate lead screw 30003. In an aspect, when coupler 30008 travels linearly along lead screw 30003, endstop detector 521, mounted on bar 30010, detects when coupler 30008 reaches a pre-selected point of travel, and endstop detector 521 informs the controller to reverse the motor 513, which reverses the direction of lead screw 30003. In an aspect, the status of the device is displayed at least by a pattern of LEDs on status display 505. In an aspect, when coupler 30008 is pushing the syringe plunger 516 into the syringe 503, the status is displayed in a first color pattern. A second color pattern can be displayed when the coupler 30008 is moving away from the syringe 503. When device 20001 is experiencing issues, the status is displayed in a third color pattern. Several states can be represented by a combination of colors, or a numerical status indicator, for example. In an aspect, status is provided through audio means, numeric/coloric means, video means, haptic means such as vibrator in the handle), and/or olfactory means. In an aspect, as cells are moving out of syringe 503 and into an organ, the pressure exerted on the cells is measured by pressure sensor 501. In an aspect, pressure sensor 501 provides sensor data to an onboard controller (not shown) that adjusts the delivery force to maintain a desired pressure. In an aspect, adjusting the delivery force includes adjusting the rate at which lead screw 30003 is laterally progressing through adjusting the rotation speed of motor 513. In an aspect, pressure sensor 501 is located against the syringe 503. In an aspect, pressure sensor 501 is wired to battery 523 and a controller, or communicates data wirelessly. In an aspect, other characteristics besides pressure of the cells are measured by other sensors positioned appropriately on the device and in proximity to the cells. In an aspect, thermal, pH, and dissolved oxygen data are collected and stored. In an aspect, data storage is remote to device 20001, and data are transmitted wirelessly to a remote (to device 20001) storage location.

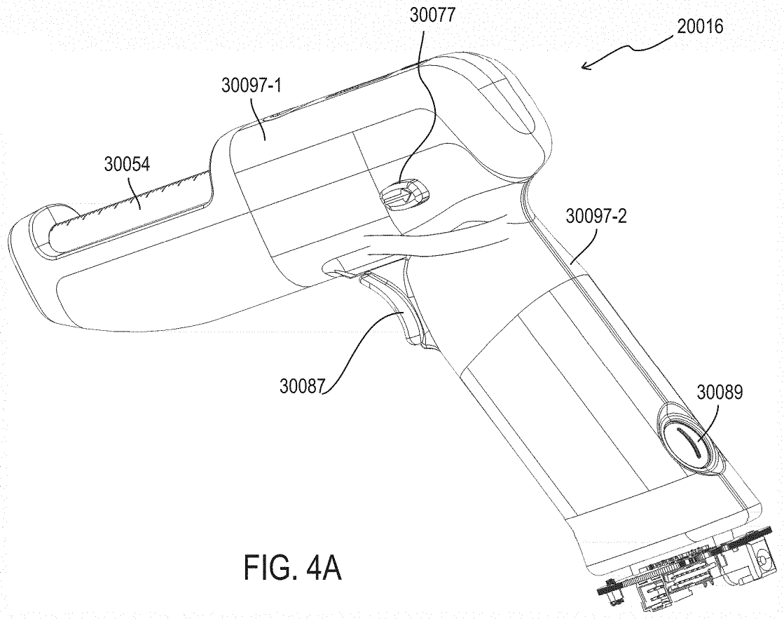

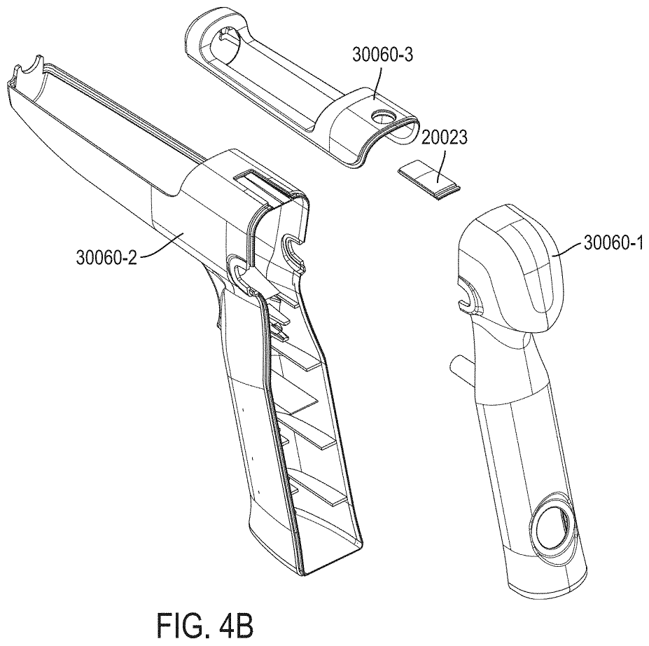

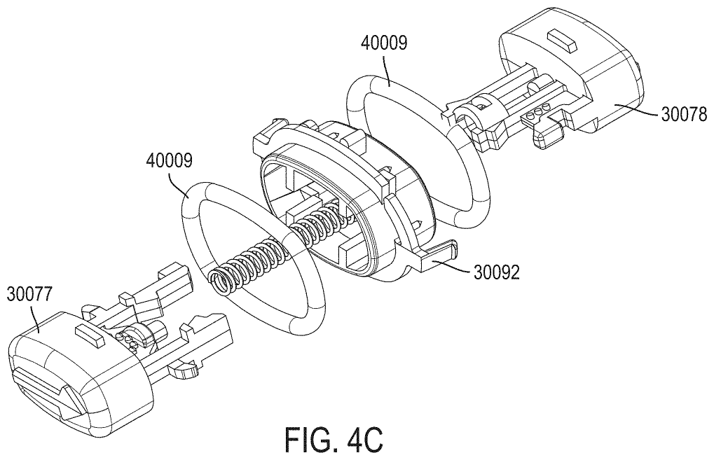

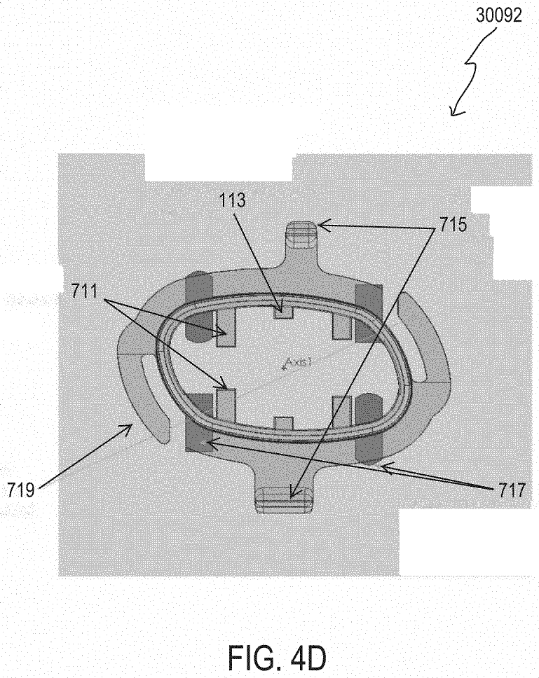

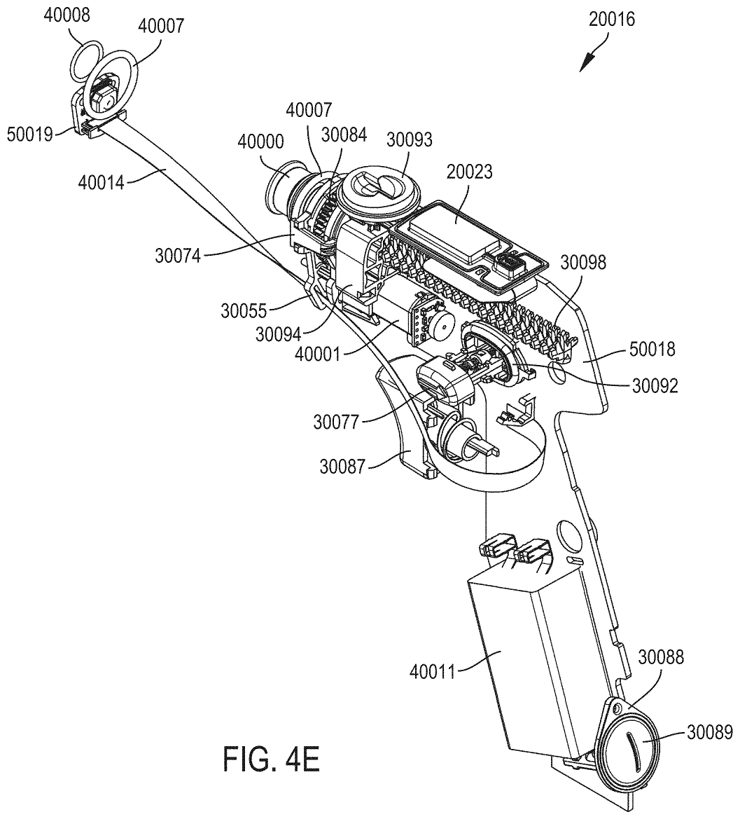

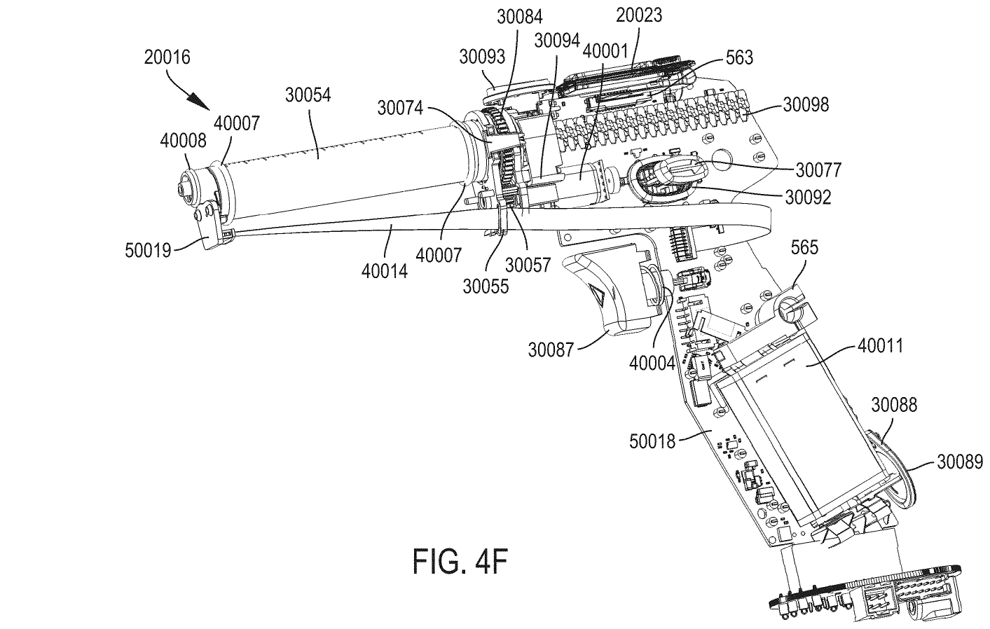

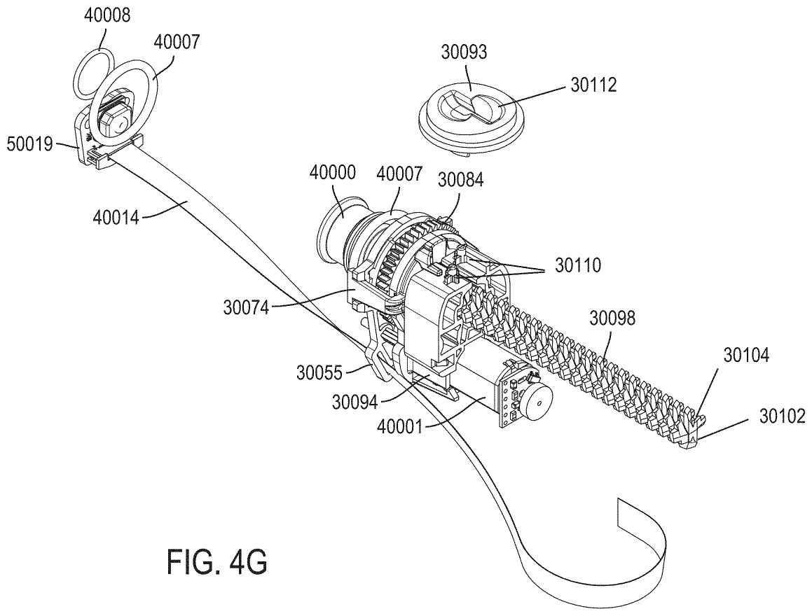

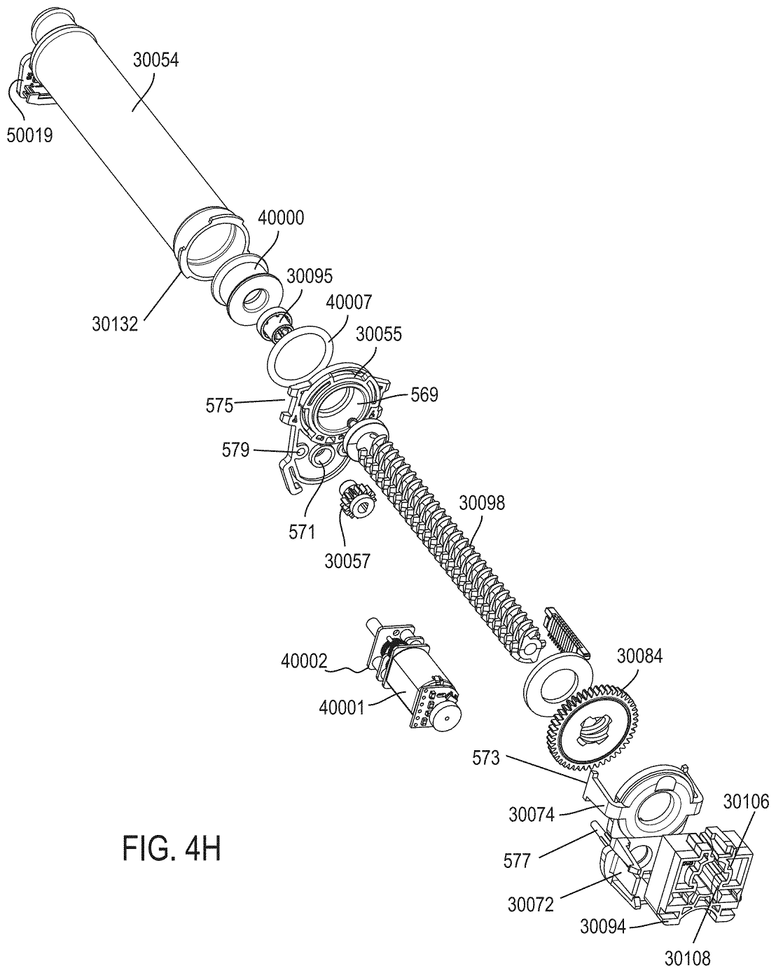

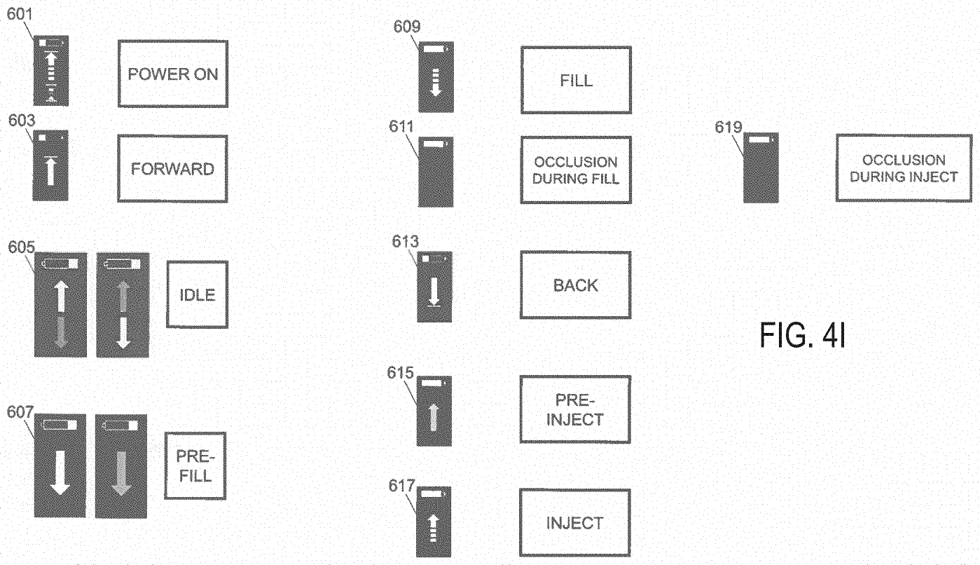

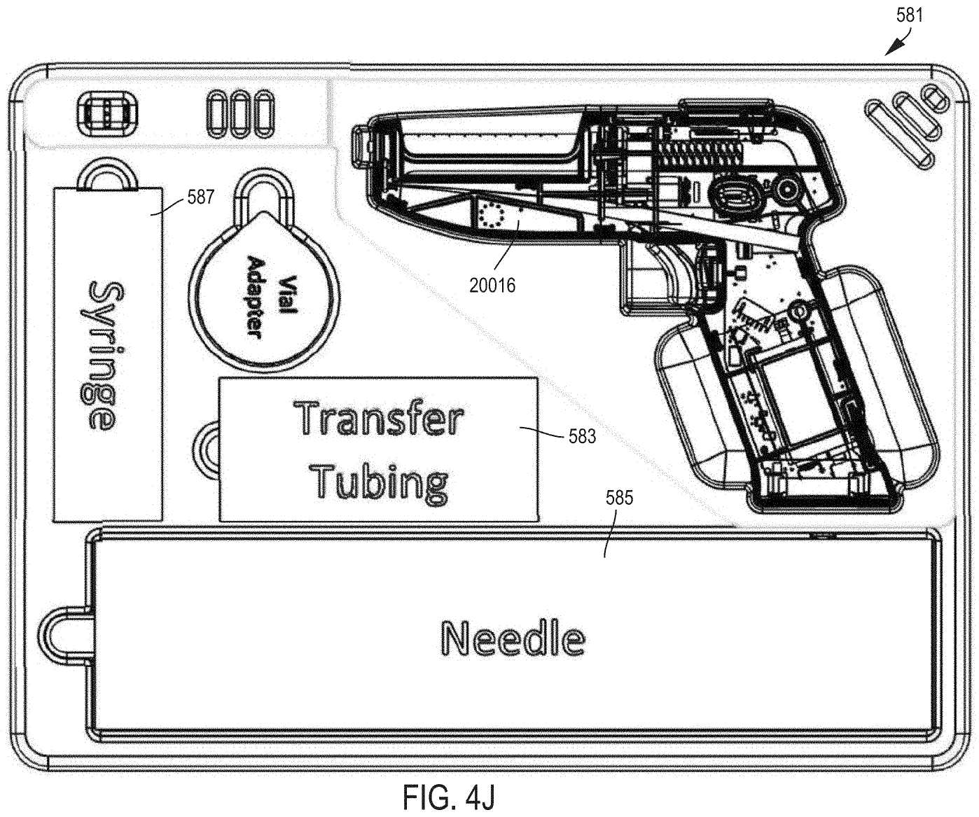

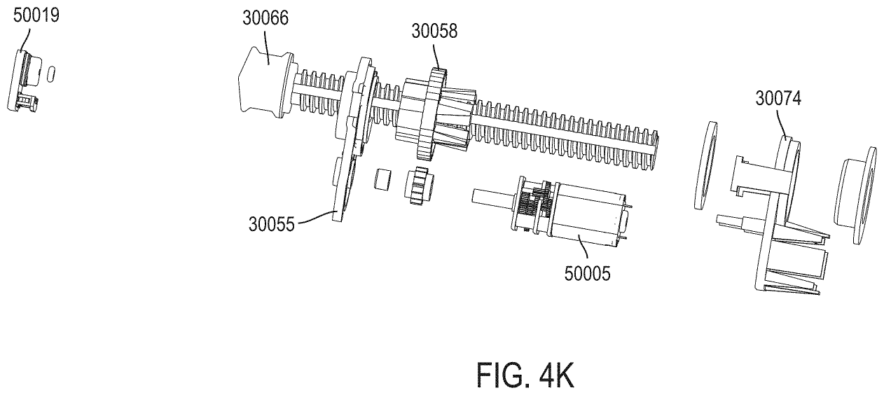

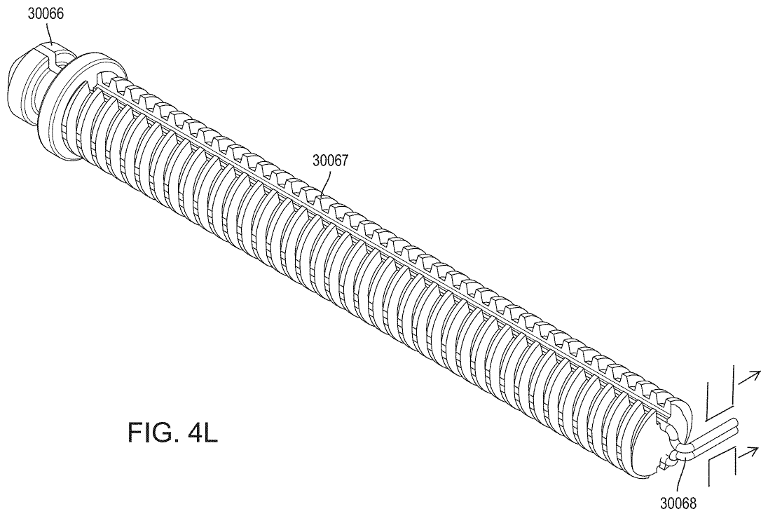

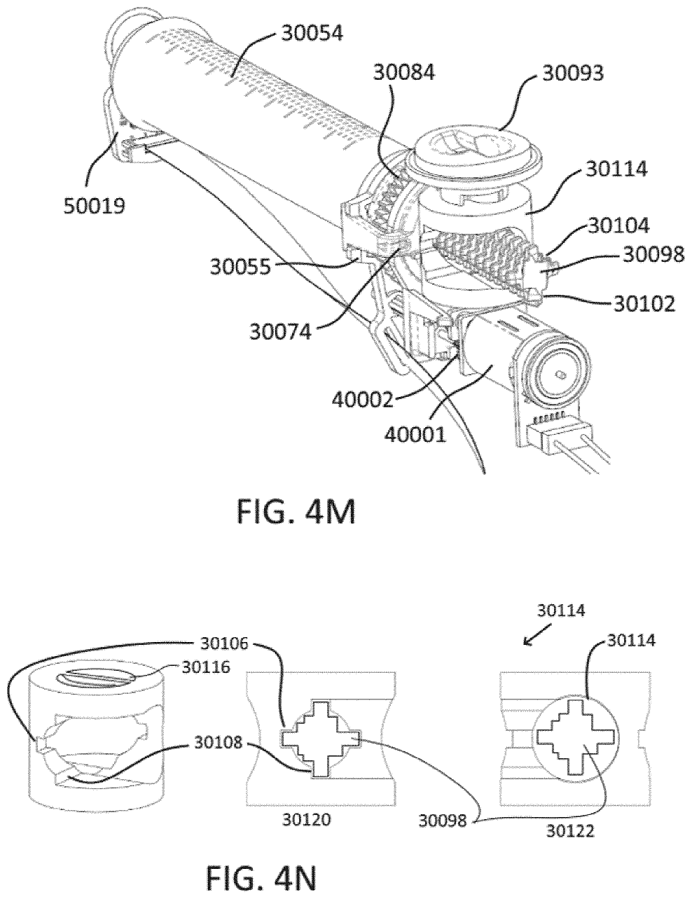

Referring now to FIGS. 4A-4L, another exemplary configuration of the electromechanical injection device of the present teachings is shown. In an aspect, device 20016 includes two-part case 30097 having right side 30097-2 and left side 30097-1 that are operably coupled to surround the components of device 20016. In an aspect, case 30097 includes mounting cavity 567 (FIG. 4B) for syringe 30054. Case 30097 can be formed to accommodate various sizes of syringe, and can include an adapter (not shown) that enables the cavity to accommodate a syringe that is smaller than the cavity. In an aspect, a case can include more than two sections. For example, case 30060 (FIG. 4E) includes rear section 30060-1, mid-section 30060-2, and syringe section 30060-3. Other cases are contemplated by the present teachings. The main function of the case is to protect the components from environmental contaminants. In an aspect, the case provides a sturdy grip for the user to steadily deliver cells to the organ until the delivery is complete or otherwise terminates. In an aspect, the case provides access to charging and data ports. In an aspect, the case provides access to a power supply, and access to a switch associated with the power supply. In an aspect, the case provides access to an activation means that enables the injection process. In an aspect, the activation means is a trigger. In an aspect, the case provides a way for the user to monitor the status of the device and possibly the status of cell delivery, among other things.

If the device ceases to function, the unused or residual cellular mixture can be recovered using the recover knob 30093 and one of the disclosed anti-rotation flexures 300094, 30114. The cam can allow the lead screw to be driven to allow liquid to be pulled out by another syringe. Allowing the tip or the plunger seal to rotate on the end of the lead screw reduces friction during recovery of the cellular mixture.

Referring now to FIG. 4E-4H, the shown configuration includes a syringe/lead screw system coupled with an override actuator cam that can allow the lead screw to be driven to allow liquid to be pulled out by another syringe. The configuration includes a PCB into which the trigger mechanism, a user display, a trigger button/snap action/flex snaps, a direction selection button, and a power supply are coupled. The PCB can include mountings and connections between the master processor and devices that are supplying data to the master processors. Communications interfaces can also be included on the PCB.

Referring now to FIGS. 4E-4H, an exemplary set of components that enables electromechanical organ injection are shown. The general categories of components include a battery 40011, an activation mechanism 30087, a motor 40001, a drive mechanism 30057, 30087, 30094, and a delivery mechanism 30098, 30054. In an aspect, the battery 40011 is mounted to a PCB 50018 with mounting springs 565. Other forms of power are contemplated by the present teachings such as grid power, capacitors, supercapacitors, rechargeable batteries, single use batteries, and various types of batteries including lithium, alkaline, carbon zinc, silver oxide, and zinc air batteries, for example. In an aspect, power switch 30089/battery button pressure plate 30088 activate/deactivate the battery power supply, or place the battery in a low power mode, to preserve the life of the battery between uses, for example during a long procedure. In an aspect, power is supplied by a 9V battery. In an aspect, the activation mechanism is trigger 30087.

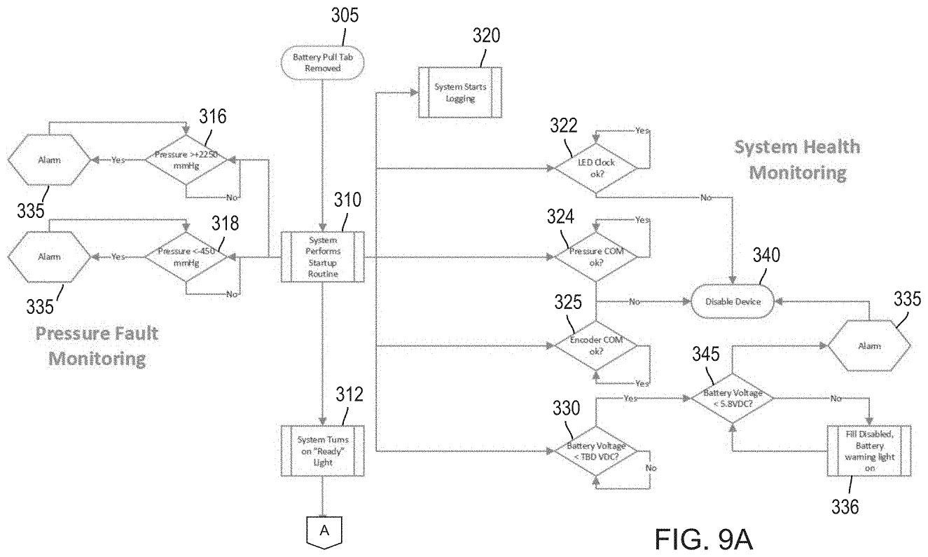

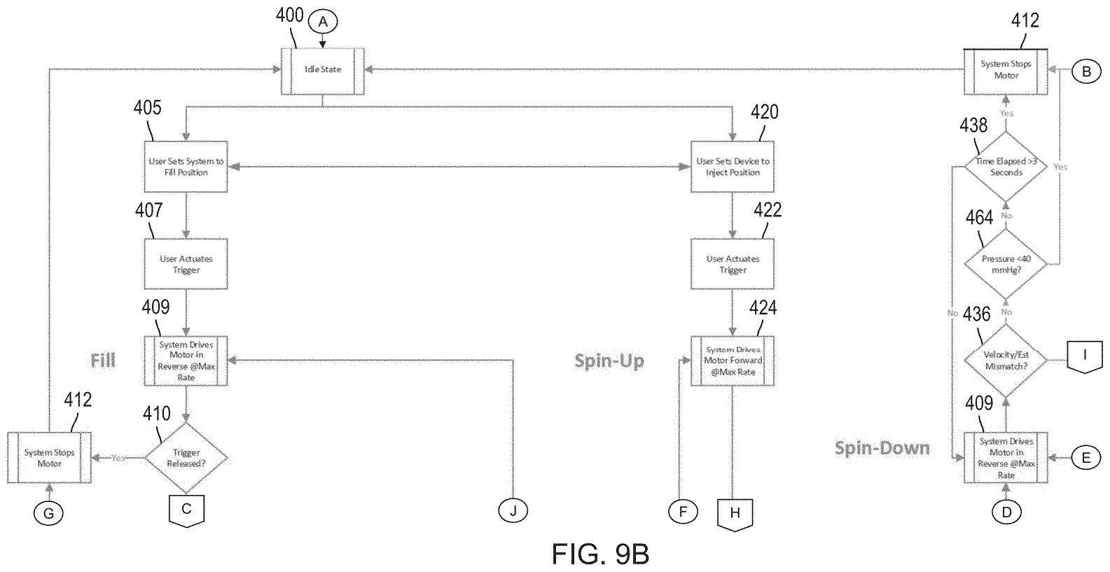

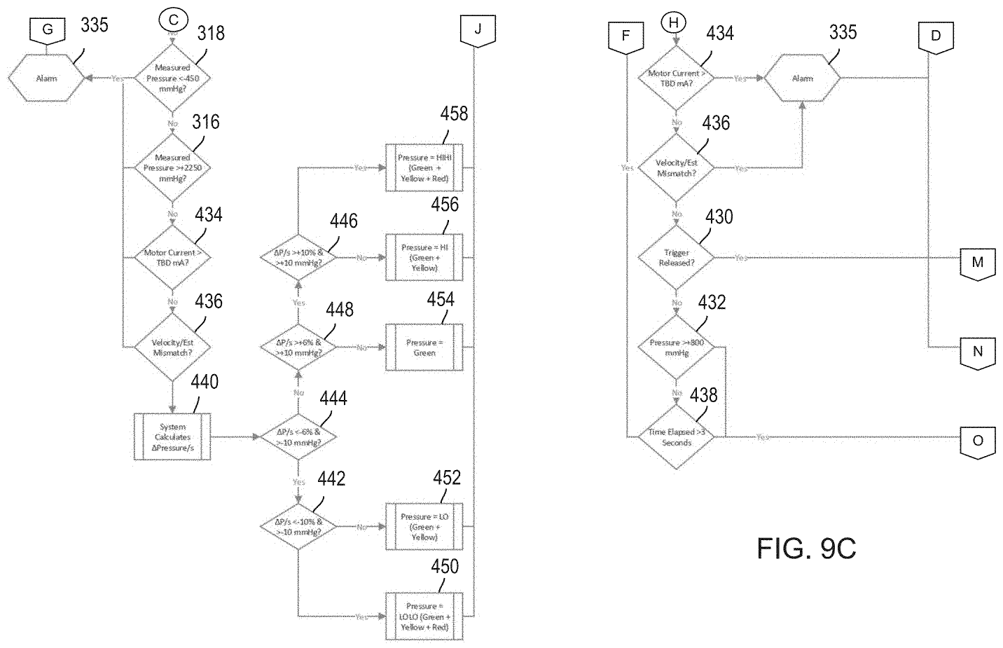

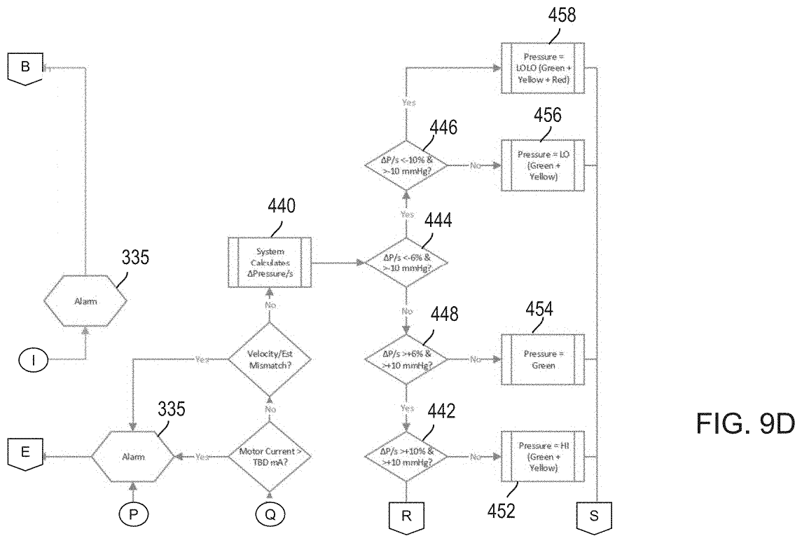

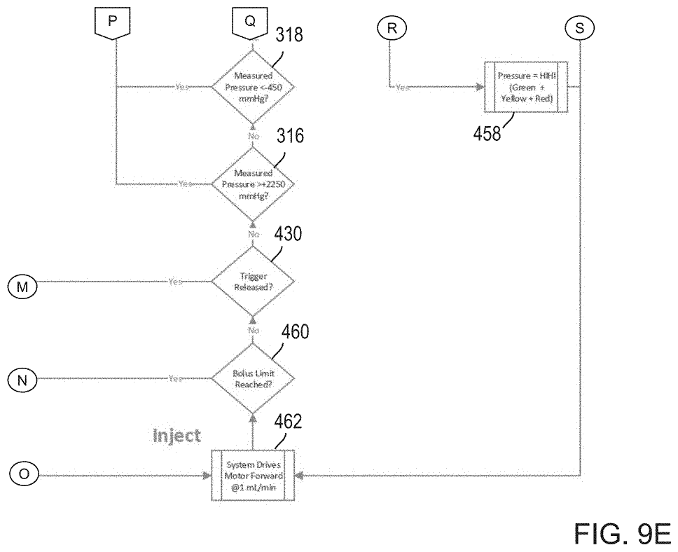

In an another aspect, the batter switch 30089 powers the low power circuit that powers a latching relay. The first time the trigger 30087 is fully pulled toward the handle, the latching relay is closed and connects the battery power to the controller located on the PCB 50018. Continued or subsequent pulls on the trigger 30087 command the controller to drive the motor. The speed and direction of the motor depends on the measured pressure from pressure sensor 50019 and the control logic described below and referring to FIGS. 9A-9E. In an aspect, trigger 30087 is operably coupled with spring 40004, enabling trigger 30087 to return to its pre-depression position. Other forms of activation are contemplated by the present teachings. Device 20016 can include, for example, a switch or a push button that can activate motor 40001. In an aspect, device 20016 includes a communication means (not shown) in which remote activation/deactivation is possible. In an aspect, the remote activation message is communicated through wireless or wired means.

Continuing to refer to FIGS. 4E-4H, the motor turns the drive mechanism to apply force on the delivery mechanism which includes a lead screw/plunger 30098 and syringe barrel 30054. In an aspect, motor 40001 rotates pinion 30057 that in turn rotates gear 30084 which includes a medium helix bore gear. The helix bore gear of gear 30084 rotates around lead screw 30098, which translates forward or backward depending on the direction of rotation of gear 30084. Anti-rotation flexure 30094 prevents the lead screw 30098 from rotating, thereby causing the rotation of the bore gear 30084 to translate the lead screw forward or backward along the longitudinal axis of the lead screw. Lead screw 30098 is capped on its forward end by flex tip 30095 (FIG. 2H) and plunger seal 40000. Plunger seal 40000 (FIG. 4K) communicates directly with the cellular mixture in syringe barrel 30054, and pushes the mixture forward and out of syringe 30054 as the lead screw 30098 moves forward. O-rings 40007/40008 (FIG. 4G) seal the housing 30060-2, 30060-3 (FIG. 4B) to the syringe 30054, thereby protecting the components inside the housing 30060-1,2,4 from the environment. Pressure measurements from sensor 50019 are transmitted to a controller on circuit board 50018. In an aspect, the controller can use the pressure information to create indicators for display 20023. In an aspect, the controller can use the pressure information to adjust the speed of lead screw 30098, for example. In an aspect, syringe 30054, motor 40001, and lead screw 30098 are operably coupled by a combination of gearbox front plate 30055 and gearbox back plate 30074. Gearbox plate 30055 includes coupling cavity 569, which also acts as the bearing surface for the hub on gear 30084, that enables plunger seal 40000, installed in syringe 30054 during assembly, to move as drive gear 30084 rotates and lead screw 30098 moves linearly. Gearbox plate 30055 provides motor shaft termination cavity 571 (FIG. 4H) and a surface against which pinion 30057 rotates. In an aspect, to limit rotation to the movement of lead screw 30098 and the gears, syringe gearbox back plate 30074 operably couples with gearbox plate 30055 by coupling connecting tabs 573 (FIG. 4H) with tab slots 575 (FIG. 4H) and pins 577 (FIG. 4H) with cavities 579 (FIG. 4P). In an aspect, back plate 30074 includes tabs and hooks 30072 that guide and secure the motor 40001 to the back plate 30074. In an aspect, various designs of back plate 30074 can accommodate various gearbox sizes. Other geometries of back plate 30074 and gearbox plate 30055 are contemplated by the present teachings. In an aspect, back plate 30074 includes a guide 30055 to rout of flat cable 40014 by the drive gear 30084. Back plate 30074 includes pinchers 30072 that hold the syringe/gearbox assembly in place in the case during assembly.

Referring now to FIG. 4F, the pressure applied to the cellular mixture in the syringe 30054 may be measured in several ways. In an aspect, the cellular mixture pressure in the syringe may be approximately measured by the force exerted on the pressure sensor 50019 by the syringe barrel 30054. Syringe barrel 30054 comprises a nozzle and luer fitting 40008 that located off the center line of the barrel to provide a larger contact area for the pressure sensor 50019. This design is advantageous as the barrel directly contacts the sensor plate or sensor element on the pressure sensor 50019 without sliding or rotating elements between that may add friction and reduce both the accuracy and responsiveness of the signal to changes in the mixture pressure inside the syringe barrel 30054.

In another aspect, the mixture pressure may be measured with a pressure sensor 501 (FIG. 3C) that contacts a pliable section of tubing downstream of the syringe 503. In another aspect the mixture pressure may be measured with a force or pressure sensor mounted between the drive mechanism element such as the back plate 30074 and the housing 30060-1, 30060-2. In an aspect, the mixture pressure can be assessed by measuring the voltage and/or current supplied to the electric motor 40001.

### Flexures to Allow Cellular Mixture Recovery

Referring now to FIGS. 4F-4G, in a first position shown in FIG. 4F, the anti-rotation flexure 30094 prevents the lead screw 30098 from rotating axially about it longitudinal axis. The lead screw 30094 includes one or more flat surfaces 30102 that are parallel to the longitudinal axis. The flexure 30094 has a matching flat surface 30108 that prevents the lead screw 30098 from rotating. Additionally or alternatively, the lead screw 30098 may one or more axial notches 30104 and the flexure 30094 may have matching insert 30106 that that prevents the lead screw from rotating. The combined action of the rotating gear 30084 and the anti-rotation flexure 30094 results in the lead screw 30098 translating along its axis without rotation.

In normal operation, residual cellular mixture is recovered by the delivery device injecting the fluid back into a vial for storage. In some cases, the delivery device is unable to deliver the residual cellular mixture due to pump failure, low battery etc. In these cases, the residual cellular mixture can be recovered by manually driving the lead screw forward and pushing the cellular mixture out of the syringe barrel 30054. In the delivery configuration, manually pushing the lead screw forward would require a significant force to back drive the gear 30084, pinon gear 30057 and electric motor 40001.

In one alternative, the anti-rotation flexure 30094 is spread open by rotating the recovery knob 30093. The recover knob 30093 includes protrusions that interface the elements 30110 at the top of the anti-rotation flexure 30094 on each side of the split. Rotating the recover knob 30093 applies force on the flexure protrusions 30110 that spread the two halves of the anti-rotation flexure 30094. The spread-opened flexure 30094 moves the surfaces 30106, 30108 away from the lead screw 30098, which allows the lead screw 30098 to rotate. Once the lead screw 30098 is free to rotate, the lead screw 30098 can be manually translated forward without turning the gear 30084 and thus reduces the force required to push the lead screw 30098 forward and push the residual cellular mixture from the syringe barrel 30054. The recover knob 30112 may include a one way drive slot 30112 that allows the recover knob 30093 to used only once. After use, the recover knob with the one way slot 30112 cannot easily be returned to a delivery configuration which would allow the anti-rotation flexure 30094 to re-engage the flat surfaces of the lead screw 30098. The one-way drive slot 30112 on the recover knob 30093 discourages a user from using the device after a failure.

Referring now to FIGS. 4K, an exemplary configuration of syringe/lead screw device 20015 is shown. Device 20015 includes a manual override system in order to recover cellular mixture when the device has been disabled electrically or programmatically. Specifically, rather than rigid threads in the bore of gear 30058 to convert the rotary motion of motor 50005 to linear motion of lead screw 30098, internal threads of gear 30058 can move in/out of engagement with lead screw 30098. In an aspect, multiple flexible petals that form both the bore and a hub on gear 30058 are used. The natural, unconstrained shape of the petals is in the flexed-out position, where lead screw 30098 can slide freely through the bore of gear 30058. When the petals are constrained radially, the thread cut into the inside surfaces of the petals engage with lead screw 30098. An exemplary configuration includes a removable rigid ring (not shown) that radially constrains the petals that that they engage with the threads on the lead screw. In another configuration, an O-ring (not shown) constrains the petals. In yet another configuration, a u-shaped piece (not shown) slides through the housing to constrain the top and the sides of the petals. In all these configurations, the user would remove the constraint before manually pushing the lead screw forward to recover the cellular mixture.

Referring now to FIGS. 4M 4N, an exemplary configuration of a system to allow manual recovery of the cellular mixture comprises a round anti-rotation flexure 30114 that rotates between two positions: 30120 where the flat surfaces of the lead screw 30098 are engaged and 30122, where the inner surface of the flexure 30114 allows the lead screw 30098 to rotate. In the delivery position 30120, the round anti-rotation flexure 30114 engages with at least one of a flat surface on the side of the lead screw and a longitudinal notch on the lead screw, thus preventing rotation of the lead screw 30098. In a second position 30122, the round anti-rotation flexure 30114 allows the lead screw 30098 to freely rotate. The round flexure 30114 rotates between the delivery position 30120 and the second position 30122 about an axis perpendicular to the lead screw longitudinal axis. The round flexure 30114 has a rounded exterior so that it can rotate, while remaining in a fixed location relative to the plate 30074. The round anti-rotation flexure 30114 may include recesses 30116 that permit engagement with the recover knob 30093 and allow user to change the position of the round flexure 30114 by rotating the recover knob 30093,

In an aspect, device 20015 can include a detachable tip plunger which can allow tip 30066 to detach from lead screw 30067 (FIG. 4T) and move independently. In an aspect, wire clips 30068 (FIG. 4U) are used to detach tip 30066. In an aspect, a quick release pin style design (not shown) is used to detach tip 30066. In some configurations, a back-drivable lead screw with relatively high pitch lead screws can be back-driven by applying axial force. In some configurations, the gear train can be decoupled, which can move the motor axially to move gears out of mesh and allow free rotation of the driven gear. In some configurations, for example in injection device 20016 (FIG. 4H), anti-rotation can be removed. A torque applied to the screw counters the friction component of the conversion of motor torque to linear plunger force. Removal of this reaction torque allows rotary motion (and therefore linear motion) independent of gear motion.

### Direction Switch

Referring now to FIGS. 4C-4E, in an aspect, the lead screw 30098 can linearly travel in forward and reverse directions by reversing the rotation of motor 40001 and thus the gear train. In an aspect, the direction of travel of lead screw 30098 can be selected. In an aspect, the selection is accomplished by use of direction selector buttons 30077/30078. The buttons cooperate through the operable coupling between the geometry of buttons 30077/30078 and selector tunnel 30092. The two buttons 30077 and 30078 are connected to each other and have sliding surfaces that ride on the guide tabs 711 in selector tunnel 30092. The buttons/tunnel assembly is configured as a bi-stable switch, so that buttons combination is at one of one of two extreme positions. The position of the buttons is determined optically by emitters and sensor mounted on the PCB 50018 (FIG. 4F). Windows in the selector tunnel 30092 allow light from at least one IR emitter on the PCB to reach an aligned IR sensor on the PCB by passing through windows/lightpipes/lenses 717 in the selector tunnel 30092. At least one IR emitter or IR sensor will be blocked by the buttons 30077, 30078 in one of the bi-stable position. At least one IR emitter or IR sensor will be unobstructed by the buttons 30077, 30078 in one of the bi-stable positions

In an aspect, other selector button configurations are contemplated by the present teachings. Referring to FIG. 4D, four exterior tabs 711 provide guidance to buttons. Center tabs 713 provide spring alignment during assembly. Mismatched sizes 715 ensure proper orientation during assembly. Spring arms 719 provide preload against the PCB to ensure proper seating. O-rings 40009 seal the tunnel to the case halves.

In an aspect, other selector button configurations are contemplated by the present teachings Referring to FIG. 4D, four exterior tabs 711 provide guidance to buttons. Center tabs 713 provide spring alignment during assembly. Mismatched sizes 715 ensure proper orientation during assembly. Spring arms 719 provide preload against the PCB to ensure proper seating. O-rings 40009 seal the tunnel to the case halves.

### Pump in Tray

Continuing to refer to FIG. 4J, in an aspect, carrying case 581 includes cavities sized to fit an exemplary configuration injection device 20016 (FIG. 4A). In an aspect, carrying case 581 includes syringe storage 587, needle storage 585, and transfer tubing storage 583, in addition to storage for device 20016. The carry tray 581 provides a convenient assembly of parts to be sterilized and then sealed. In an aspect the tray and the components in the tray may be sterilized with ethylene oxide Other configurations of a carrying case are contemplated by the present teachings. For example, the case can include a storage location for a spare battery, spare sensors, spare displays, and other items.

### Display Screens

Referring now to FIG. 4I, exemplary monitoring screens are shown. In an aspect, power button 30089 and trigger button 30087 accept user input. Other user input features are contemplated by the present teachings. For example, arrow buttons (not shown) can enable the user to adjust the pressure of the cells entering the organ. In an aspect, when power button 30089 is depressed, identifying information about the device is displayed, for example, the vendor and the version number of the device, on display assembly 20023 (FIG. 4H), powered through connector 563 (FIG. 4H). In an aspect for training, for example, a first operational screen is displayed and from then on, screens can be stepped through by repeatedly pressing trigger button 30087 (FIG. 4H) or by pressing and holding trigger button 30087 (FIG. 4H). In an aspect, as shown in FIG. 4F, at the top of the screens is a battery level indicator. When the user has stepped through the screens, the user is returned to the first operational screen and the battery indicator is updated. In an aspect, power on self-test screen 601, a first operational screen, is displayed after the identification information screen is shown, after power-up. An arrow, possibly animated, on power on screen 601 indicates syringe motion as the device prepares for a first “fill” operation. Forward end-stop indicator display 603 indicates that the syringe is at the full forward position and thus empty. Idle display 605 indicates that the device is idle and waiting for a direction (fill or inject) operation to be selected. In an aspect, a screen switches between an arrow pointed forward and an arrow pointed backward. Pre-fill screen 607 indicates that the “fill” direction has been selected. In an aspect, depressing trigger button 30087 (FIG. 4H) starts a “fill” operation. In an aspect, an arrow toggles between two pre-selected colors. Fill screen 609 indicates that a “fill” operation is in progress. An arrow, possibly animated, indicates the direction of the syringe motion as it fills. Occlusion during fill display 611 indicates that a “fill” operation has been interrupted by an occlusion. A geometric shape such as a triangle can toggle between visible and erased in the display. Back end-stop indicator display 613 indicates that the syringe is at the full rearward position and thus full. Pre-inject display 615 indicates that the “inject” direction has been selected. In an aspect, trigger button 30087 (FIG. 4H) starts an “inject” operation. An arrow can toggle between two pre-selected colors in the display. Inject display 617 indicates that an “inject” operation in progress. An arrow, possibly animated, indicates the direction of the syringe motion as it empties. Occlusion during inject display 619 indicates that an “inject” operation has been interrupted by an occlusion. A geometric shape such as a triangle in the display can toggle between visible and erased.

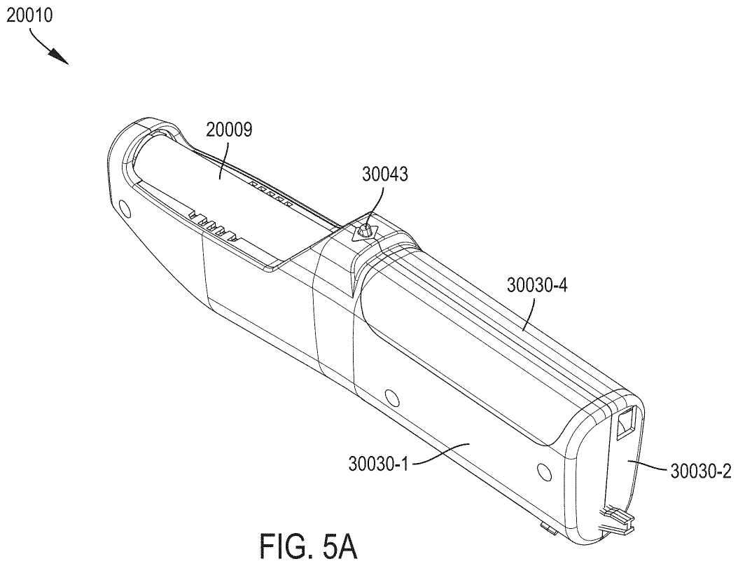

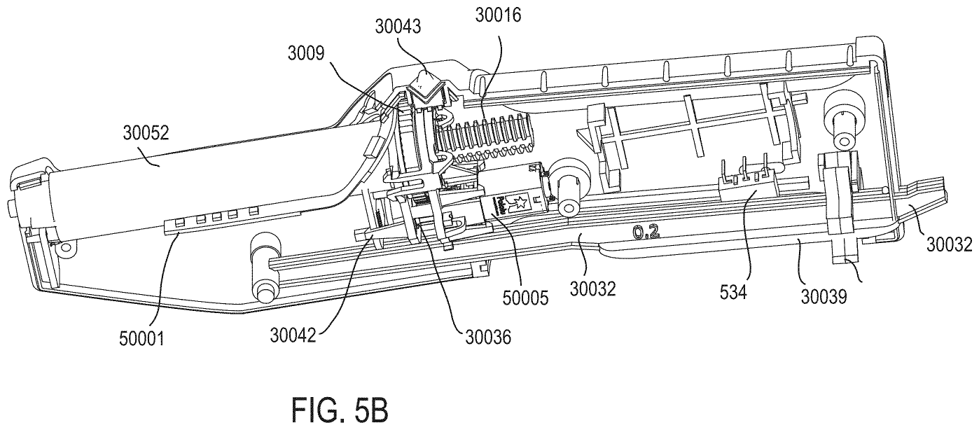

Referring now to FIGS. 5A-5B, an exemplary configuration of electromechanical injection device 20010 of the present teachings is shown. Device 20010 includes syringe assembly 20009, direction switch button 30043, bidirectional switch 541, environmental case top 30030-4, case left side 30030-1, and case right side 30030-2. Device 20010 is activated by trigger 30039. Trigger 30039 is operably coupled with trigger paddle 30032. Trigger paddle 30032 activates motor 50005 through an interaction between base snap action switch 534 when trigger 30039 is pulled and a battery (not shown). When power is provided, motor 50005 rotates motor drive pinion 30036, which in turn drives gear 30019. The threads in gear 30019 interact with the threads of lead screw 30016 to drive lead screw 30016 as it moves laterally, backwards or forwards. Switch 30043 reverses the current direction of lead screw 30025 when depressed. Gear 30019 and pinion 30036 are operably coupled with syringe 30052 and motor 50005 by the cooperating attachments of syringe gearbox plate 30042 and syringe gearbox back plate 30031. Features on syringe gearbox back plate 30031 enable the mounting of motor 50005. Features on the syringe gearbox plate 30042 couple the encoder board 50008 with motor 50005. Flex board 50001 provides directional status, pressure status, and device status through, for example, visual indicators. Other ways that status can be indicated are through haptic feedback, audible status, and plain language messages, among other ways. In operation, as lead screw 30016 is driven, cells are moved from syringe 30052 past pressure sensor 552 into an organ. In an aspect, back plate 30031 and gear plate 30042 provide mounting for the gear and lead screw assembly that includes, but is not limited to including, pinion 30048 coupled with motor 50005, j-sleeve bearing 556, and magnet 550, as well as gear 30019, thrust washer 554, and sleeve bearing 552A surrounding lead screw 30016. Device 20010 uses an encoder 30036 based on a magnetic sensor that reads the position of magnet attached to the shaft of the motor 50005 or the output shaft of the reduction gear at the end of the motor.

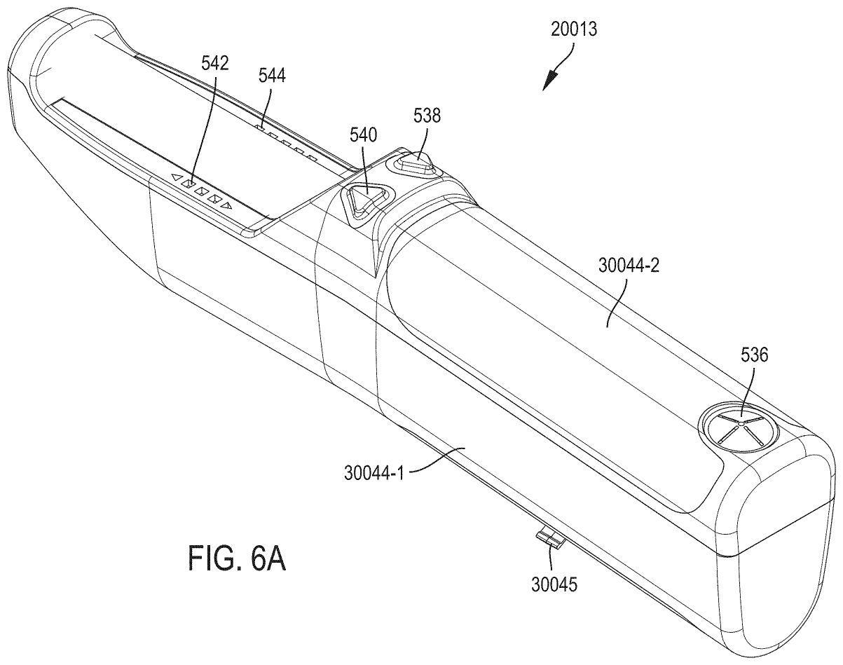

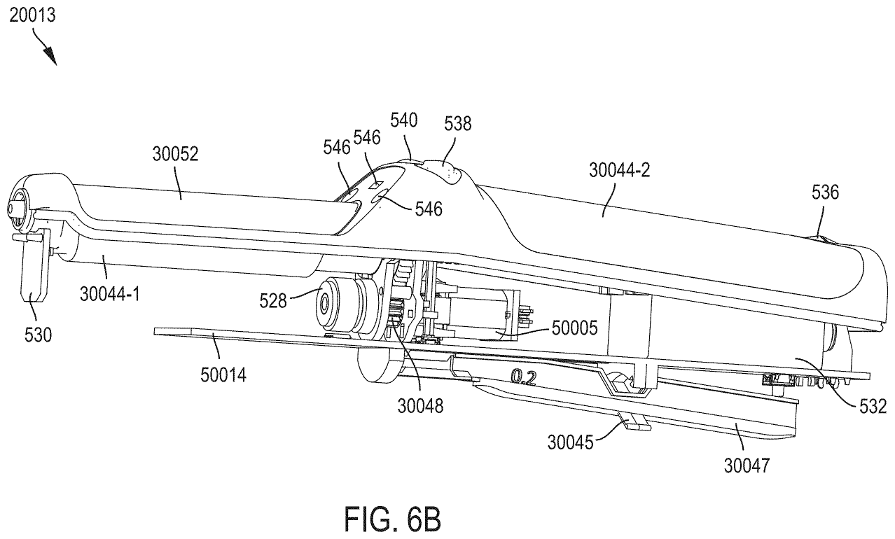

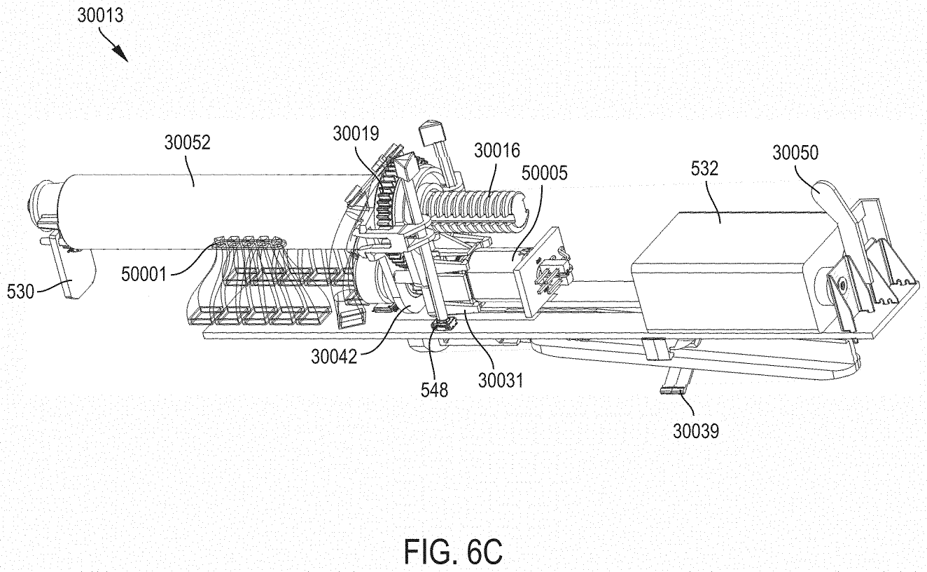

Referring now to FIGS. 6A-6C, an exemplary configuration of electromechanical injection device 20013 of the present teachings is shown. Device 20013 uses a radially magnetized magnet 528 with a pre-selected number of poles and rotated around an axis parallel to the plane of the sensor die, hence not requiring an additional PCB. The sensor integrated circuit is shown sitting on top of PCB 50014. Device 20013 includes separate forward and reverse directional buttons/switches 538/540/548, directional status indicators 542, pressure status indicators 544, system status indicators 546, and a power button 536. The environmental case of device 20013 includes case top 30044-2 and case bottom 30044-1. Case top 30044-2 includes a mounting cavity for syringe 30052. Operationally, device 20013 is powered on by depressing button 536 ( ) which is operably coupled with battery contact lever 30050 (FIG. 6E). In this way, power is supplied to the motor controller on the PCB 50014. The motor controller controls the speed and direction of the motor 50005 ( ) When, trigger paddle 30047 (is depressed, motor 50005 is activated and rotates pinion 30048 ( ) as discussed herein. In an aspect, trigger 30045 (is spring-loaded, making it return to its original position automatically. Motor 50005 ( ) rotates pinion 30048 which rotates gear 30019. Gear 30019 drives lead screw 30016, which moves laterally and pushes cells from syringe 30052 to an organ, past pressure sensor 530. An alternate configuration of the encoder of device 20013 uses a set of inductive coils to sense the position of a metallic screw. As the screw is driven, the effective position of the inductive target appears to change.

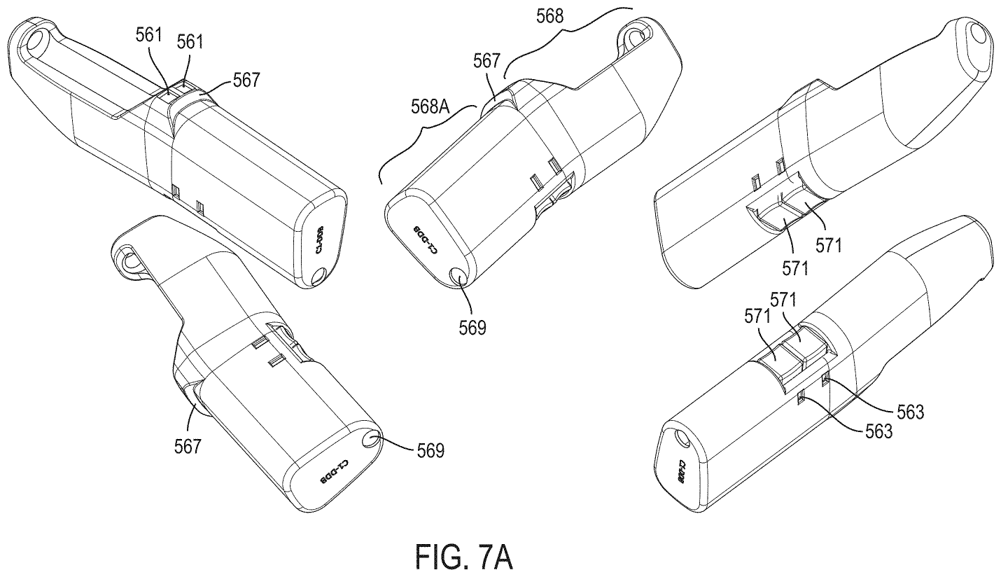

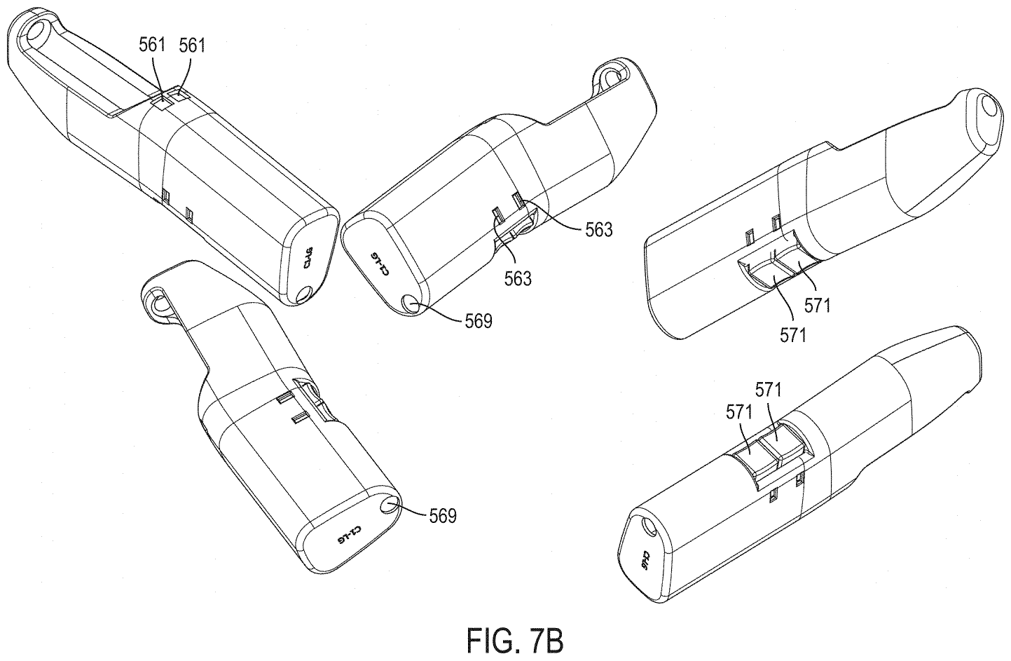

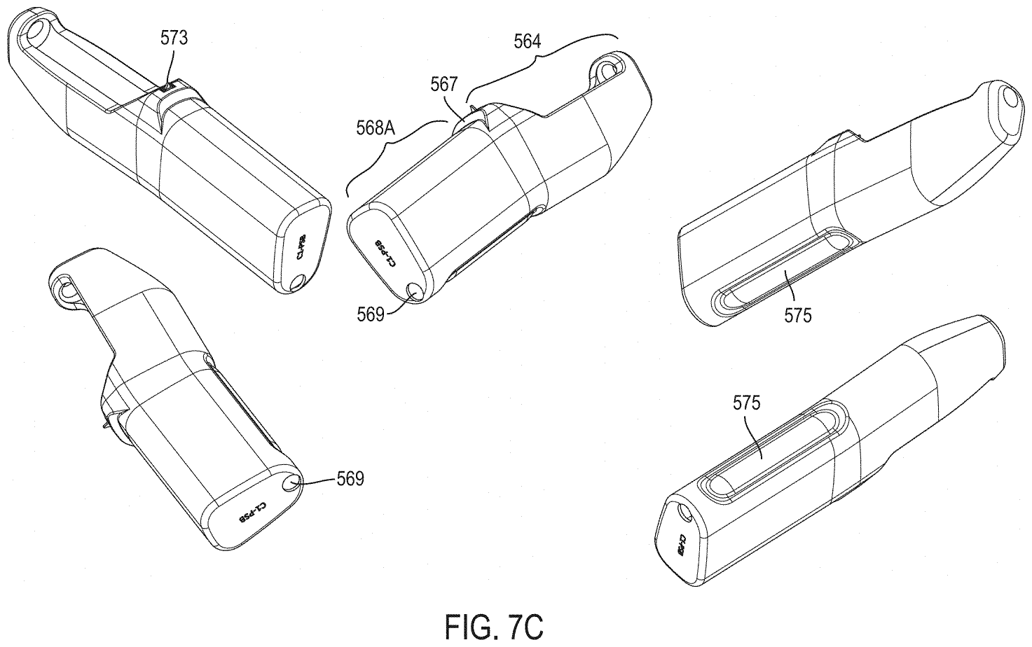

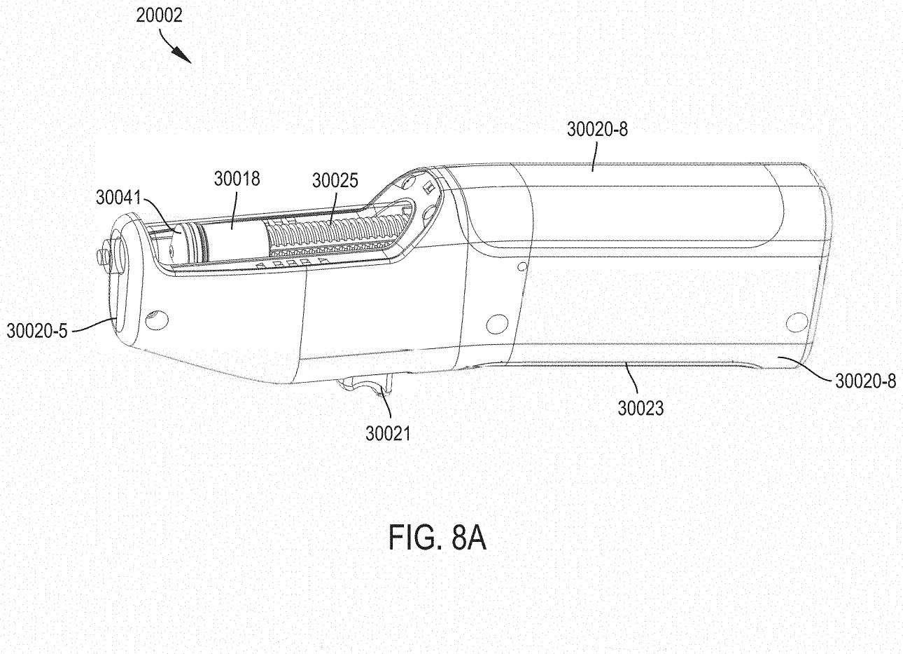

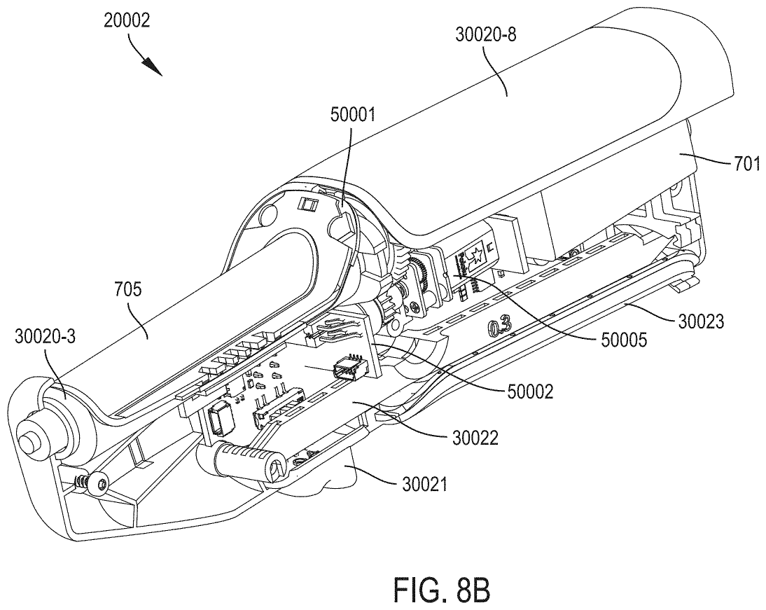

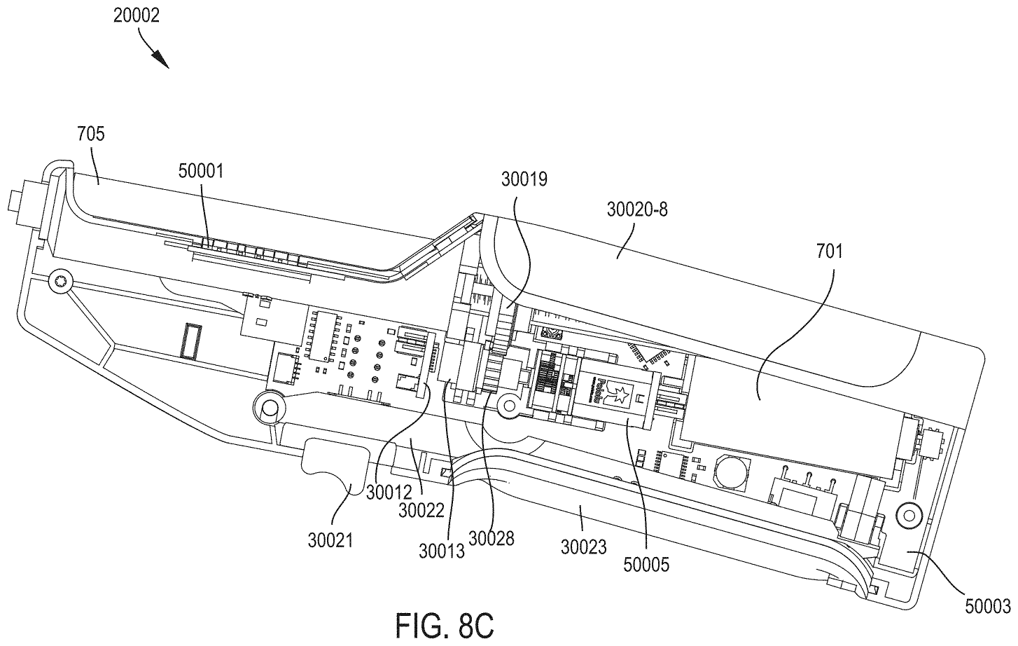

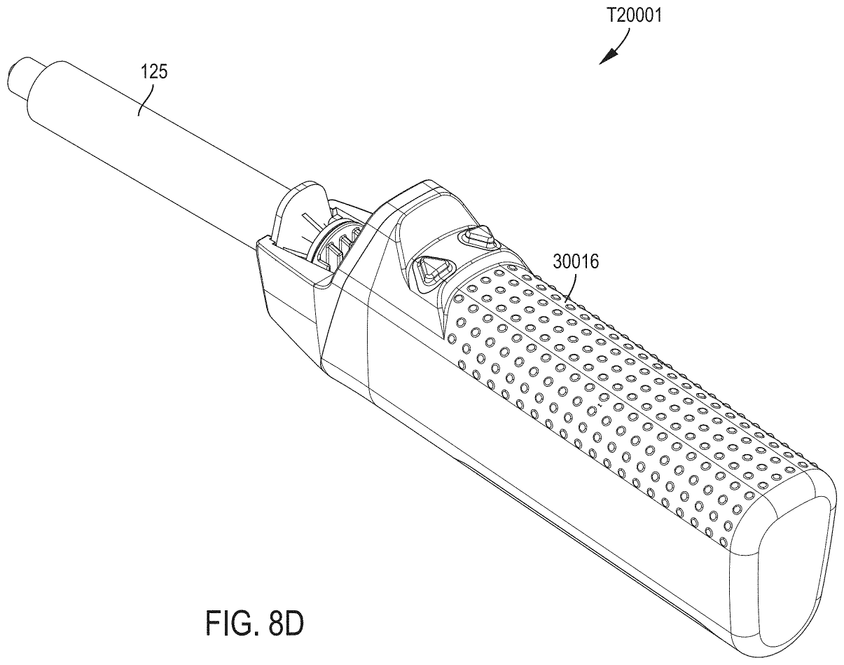

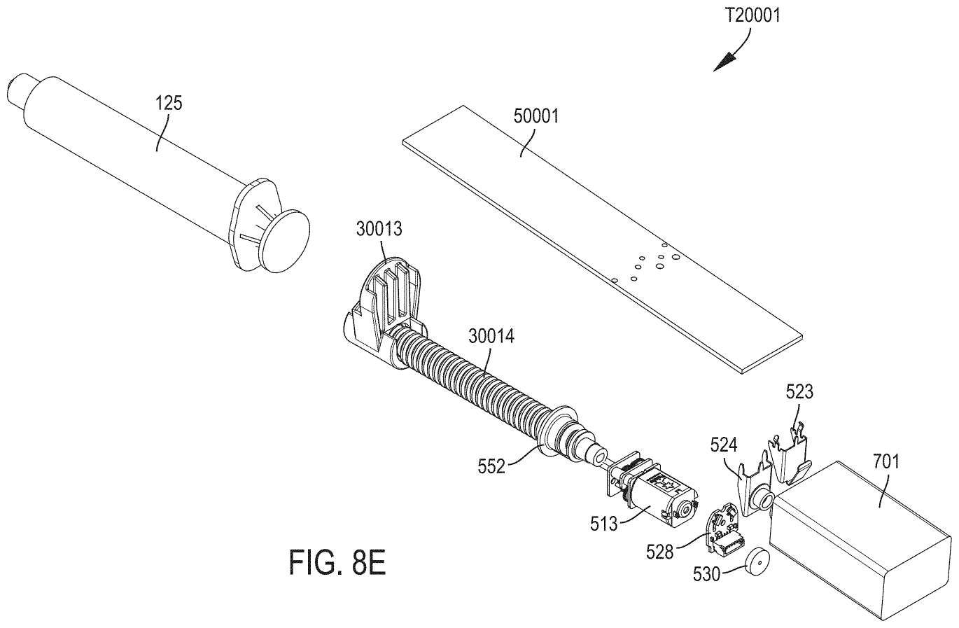

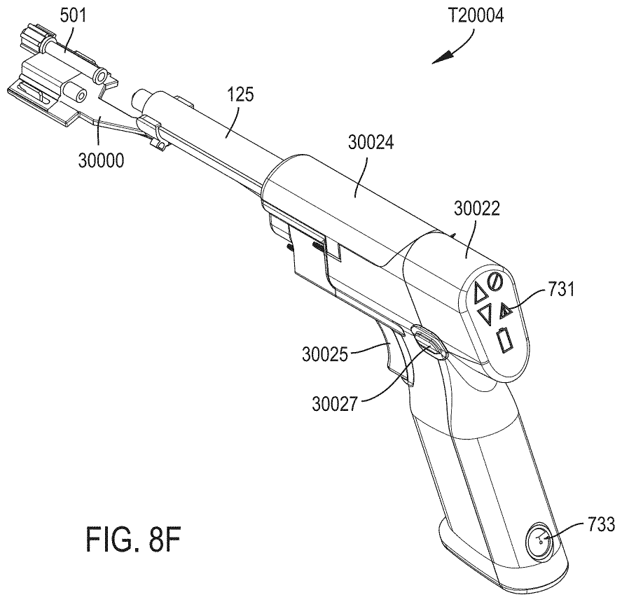

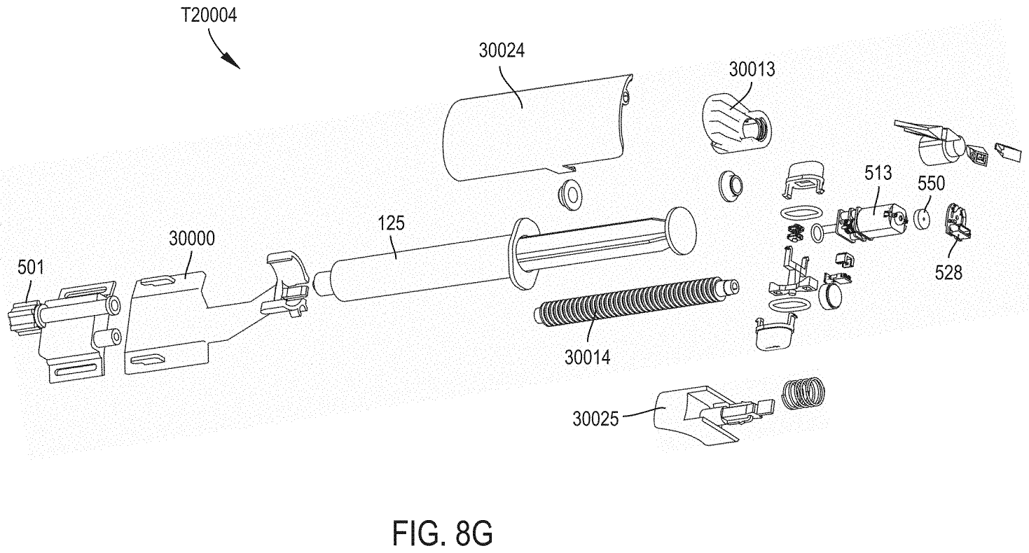

Referring now to FIGS. 7A-7C, various exemplary case configurations are shown. These cases can be combined with the drive mechanisms, sensors and internal features shown in FIGS. 4A-L, 5A-B and 6A-C. In examples shown in FIGS. 7A and 7B, push buttons 572 on the side of the case opposite the syringe cavity can be used for various types of controls. In an aspect, one button 572 is used to enable power to the device, and the other button 572 is used to disable power to the device. In an aspect, one button 572 is used to set a forward direction (towards the syringe) of the lead screw, and the other button 572 is used to set a reverse (away from the syringe) direction of the lead screw. In an aspect, one button 572 is used to raise the delivery pressure on the cellular mixture, and one button 572 is used to lower the delivery pressure on the cellular mixture. In an aspect, one button 572 is used as a trigger, and one button 572 is used to deactivate the trigger function. In an aspect, cutouts 564 as shown in FIGS. 7A and 7B are for snap features as interface options. In an aspect, status indicators are similar in construction and use to status indicators described with respect to device 20002. The status indicators can be associated with the functionality of buttons 572, or can report status about some other aspect of the system. In an aspect, cavities 561 enable mounting of directional buttons, forward and reverse. In an aspect, cavities 561 enable mounting of status indicators. Cases in FIGS. 7A and 7C have an ergonomic design where the syringe caring section 568 is at an angle from the handle section 568A. This feature reduces hand fatigue on of the user by allowing a more upright grip of the handle, while the syringe is angled downward. In the configuration shown in FIG. 7C, toggle switch 573 and activation pad 576 are shown. In an aspect, toggle switch 573, when toggled, changes the direction of the lead screw. In an aspect, when toggle switch 573 is toggled, power to the device is either enabled or disabled. Toggle switch 573 can enable other actions as well. The present teachings are not limited to the stated possibilities. The configuration in FIG. 7C includes activation pad 576. In an aspect, activation pad 576 is used to power the device. In an aspect, activation pad 576 is used to trigger action of the device. In an aspect, activation pad 576 is used to change direction of the lead screw. In an aspect, activation pad 576 is used for powering the device, triggering activities, and changing direction of the lead screw, its function being determined by the current state of the device. The syringe plunger/lead screw interface in the configuration shown in FIGS. 7A-7C is similar to the interface depicted with respect to device 20013 (FIGS. 6A-6C) The paddle trigger flex is an over-molded/elastomeric component to complete the housing while allowing the trigger to be actuated In operation, the trigger works as follows. Paddle trigger 30022 pivots, and actuates a micro switch on the PCB. The snappiness/tactility of the trigger is provided by a piece of spring steel spanning between the case halves. In an aspect, a plastic version uses a thin plastic web rather than the spring steel used in the device shown in FIG. 8E.

Referring now to FIGS. 8A-C, an exemplary configuration of electromechanical injection device 20002 of the present teachings is shown. Device 20002 includes case top 30020-8, case right side 30020-5, case left side 30020-6, syringe mount 30020-3, and paddle trigger flex 30023 that together protect the inner components of device 20002. Syringe 705 surrounds lead screw 30025, syringe plunger tip 30018, and plunger seal 30041. Electronic parts are connected to PCB 50003 which includes a processor that sequences commands that cause device 20002 to deliver the cellular mixture in syringe 705 to an organ. Flex board 50001 includes sensors and an LED board (not shown) includes status indicators reporting information collected by the sensors. When paddle trigger flex 30023 engages with paddle trigger 30022, battery 701 begins supplying power to motor 50005. When direction selector 30021 is depressed, pinion 30028 (FIG. 8B) begins rotating, causing gear 30019 to rotate, and lead screw 30025 to move laterally. In an aspect, press in pinion shaft adapter 30013A engages with pinion 30028 to couple pinion 30028, the pair of which surrounds and rotates with the shaft of motor 50005, with encoder board 50002. In an aspect, syringe assembly 20014 includes lead screw 30025 coupled with gear 30019 and fitting parts including thrust washers 30027/709 and sleeve 707. When lead screw 30025 moves laterally forward, the cellular mixture in syringe 705 are moved out of syringe 705.

Referring now to FIGS. 8D-8E, configurations are shown in which the device of the present teachings uses a commercially-available field-installed syringe. In an aspect, the devices shown in FIGS. 8D-8E include additional features to the device shown in FIGS. 3A-3C. For example, device T20001 includes a short nose, device T20002 includes an internally-threaded plunger, device T20003 includes a fully axial actuator, and device T20004 includes a variation of the signaling interface described herein. Specifically, device T20001 includes syringe plunger actuator nut 30013 that encounters the syringe plunger and depresses it when plunger screw is driven towards syringe 125. Motor/encoder/magnet 513/528/550 drive plunger screw 30014. Battery leads/spring 524/523 maintain the position of battery 701, and PCB 50001 provides mounting for electronics. Device T20002 includes long-nosed case T30019 with a status indicator. Device T20002 (FIG. 8J) includes internally-threaded plunger 30017 that rotates as broached plunger screw 30020 is driven by motor 513, driving a syringe plunger. Plunger sleeve T30018 protects internally-threaded plunger and plunger screw 30020 from contaminants. Device T20003 includes pressure sensor 501 that detects that pressure upon syringe 125 and long-nosed case 30021. Other parts, shown in exploded form in FIG. 8M, are similar to the parts of device T20002. Device T20004 includes indicator lights 731, pressure sensor 501, pressure sensor mount device 30000, and activation switch 733. Case T30022, coupled with syringe plunger cover with hall magnet 30024, protect the electronics in device T20004. Direction switch 30027 provides a way to control the direction of plunger screw 30014. Pressure sensor 501 is supported by mount 30000 that couples with case T30022. In an aspect, a bias magnet located in the rear housing component ensures a pre-selected output state of a Hall effect sensor mounted on a PCB in both the door opened and door closed states. Device T20004 includes snap action plunger trigger T30025 that activates motor 513, and thus plunger screw 30014. Vibration motor 743 provide haptic feedback, and the trigger slides a tab 741 that interrupts an IR signal and signals that the operator has actuated the trigger.

### Operations

Referring now to FIGS. 9A-9E, in an aspect, the devices shown and described herein are used according to the following process. An exemplary method of use of the system of the present teachings is shown in flowchart form. The flowchart shows a combination of user actions, mostly having to do with patient care, and actions automatically initiated by the device, for example, in pressure fault monitoring and system health monitoring. A syringe is filled to a pre-selected amount, for example, but not limited to, an amount based on the requirements of the therapy. For example, the therapy can call for a 10 mL liquid delivery. The syringe can be filled with cells manually, or can be automatically filled, possibly at a station along a manufacturing line. The cells are ready for the injection process when, for example, they have reached a desired level of confluence. Other characteristics of the cells, possibly cell or organ-dependent, can be used to determine their readiness for insertion into the organ. When the syringe is filled to a desired volume, it is inserted into the device of the present teachings and secured. The insertion/securing process can be manual or automatic. The automatic process can be enabled by a manufacturing line that is used to build the device. At a station along the line, the filled syringe can be inserted by the manufacturing line robot into the device, and the robot can secure the syringe to the device. The securing means can include, but is not limited to including, a syringe restraint. In an aspect, the syringe restraint is held in place by a turn latch. In as aspect, the syringe restraint has a latch, or has a mutual attachment mechanism such as a VELCRO® strip. Operationally, the use of the device can include the process of using a direction selector to put the device in reverse, pressing the trigger to extract the cellular mixture from vial, and releasing the trigger when complete. The process can include replacing the vial with a pressure sensor and needle set, connecting a pressure sensor cable, and using the direction selector to place device in dispense (forward) mode. The process can further include pressing and holding the trigger to initiate dispense. The motor will spin up to reach a pre-selected dispense pressure, then decelerate to a pre-selected delivery speed. The motor automatically stops when a pre-selected amount of effluent (cells) has been dispensed, or a pressure drop is detected (in which case the trigger is released and the fault is cleared), or the trigger is manually released. The motor retracts the plunger to equalize the pressure and prevent further dispensing of the effluent.

The flowchart in FIGS. 9A-9E includes the steps of starting the electromechanical device, readying the device for delivery, delivering the cells from the device, and shutting down the delivery. In an aspect, the user begins by thawing the injection solution, and removing the device from its packaging. Referring to FIG. 9A, in an aspect, a user can remove a battery pull tab 305 or other means to indicate to the device perform a startup sequence 310, and connecting the injection device to the thawed injection storage container. In an aspect, the startup sequence includes beginning logging of device and user actions 320, verifying that a clock that the device might have is operating correctly 322, verifying that the communications means providing pressure 324 and encoder data 325 are operating correctly, and determining the status of the battery 330. Under a pre-selected set of conditions, an alarm can be raised 335 and the device is disabled 340. The pressure values are checked for excessive low values 318 and a high condition If the startup routine is successfully executed status indicators are activated 312, if present.

Referring to FIG. 9B, in an aspect, the device includes an idle state 400 where processing returns when the motor is stopped. The device exits the idle state when the user takes an action, for example to fill the device 405 or begin an injection sequence 420. In an aspect, the fill process includes the user's setting the device to a fill position and the user's activating the device's trigger 407. In an aspect, the system drives the motor in reverse 409 at a pre-selected speed until the trigger is released 410 and the motor stops 412, moving the device into idle state 400. At this time, the user can remove the injection device from the injection storage container and connect the injection device to transfer tubing, and the transfer tubing to the injection needle. The injection device is set to injection position 420 and pulls the trigger 422 to initiate priming 424 at a pre-selected forward prime rate. The pre-selected forward prime rate is a different from the pre-selected reverse rate of 409. Priming is complete when either the pressure rises to a predetermined minimum, When priming is complete, the user stops the priming action by releasing the trigger and inserts the injection needle into the patient.

After priming, the user sets the device to injection position and initiates the injection action. The user waits until a pre-selected amount of solution is injected into the patient while monitoring pressure. The user stops the injection action or the system hits a deposit limit. The user repeats the injection steps if needed, then moves the needle to the next injection site if needed. The device disables itself when the injection limit is reached. At that time, the user removes the needle from the patient and disposes the device, the transfer tubing, the injection needle, and the remaining solution. (make sure there is a reverse section)

Referring to FIG. 9C, during an injection several conditions are continually checked including if the trigger is released 430, if the pressure measured by the pressure sensor, greater than another pre-selected amount 432, or if the injection duration is greater than a preselected time period such as 3 seconds 438 or if the motor current is greater than a pre-selected amount 434 or there is a velocity/estimated velocity mismatch 436, an alarm 335 is raised and the motor is stopped 412 (FIG. 9A). Otherwise, the system calculates the rate of change in the pressure 440 and tests that value to against various thresholds 442-446 for the purpose of illuminating or otherwise activating status indicators 450-458, or any form of reporting to the user. For example, if the rate of pressure change is greater than a pre-selected percentage and the pressure is greater than a pre-selected value, some form of indication that the pressure is very high is presented to the user. Otherwise, the pressure is indicated to be high. Table I provides a list of exemplary thresholds. The device continues to operate in reverse as status indicators are provided to the user until the trigger is released.

TABLE 1

| Δpressure | Pressure | Pressure indication |
|---|---|---|
| >+10% | >+10 mmHg | Very high |
| >6% and ≤+10% | +10 mmHg | High |
| >6% and ≤+10% | ≥−10 mmHg and ≤+10 mmHg | correct |
| <−10% | >−10 mmHg and ≤+10 mmHg | Low |
| <−10% | >+10 mmHg | Very low |

Referring now to FIG. 9C, if the motor current is greater than a pre-selected amount 434, or there is a velocity/estimated velocity mismatch 436, an alarm is raised. If there is no mismatch, and if the trigger is not released, and if the pressure is less than or equal to a pre-selected amount 432, and if the elapsed time is greater than a pre-selected amount 438, in an aspect, the system drives the motor forward at a pre-selected rate 462 (FIG. 9E). If the pressure is less than or equal to a pre-selected amount and the elapsed time is less than or equal to a pre-selected amount, the injection loop continues. Referring to FIG. 9E, if a bolus limit 460 is not reached and if the measured pressure is greater than a pre-selected amount 316, or if the measured pressure is less than another pre-selected amount 318, an alarm is raised 335 (FIG. 9D) and the system goes into a loop of driving the motor in reverse 409 (FIG. 9B) at a pre-selected rate until the trigger is released or the pressure is less than a fourth preselected value 464 such as 40 mmHg or the elapse time of the reverse motion exceeds a preselected time 438 such as 3 seconds. When any of the these conditions occur the motor stops 412 and the pump returns to Idle 400

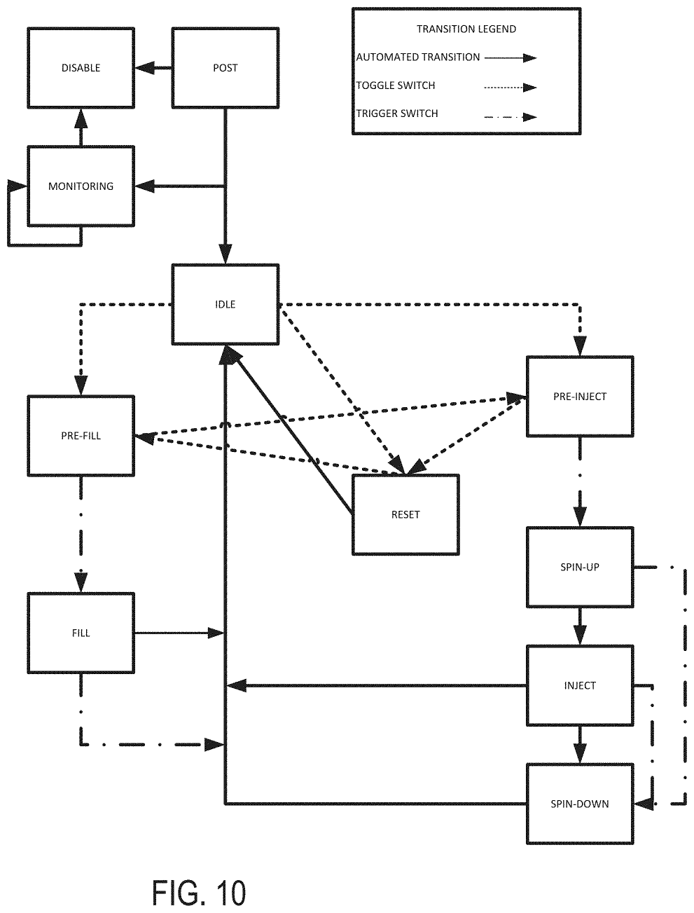

Referring now to FIG. 10, an exemplary configuration of the states of the device are shown, and legal transitions are indicated by connecting lines. Transitions can be initiated automatically, as between spin-up and inject, inject and spin-down, and fill and inject states, among others. Transitions can also be initiated by depressing the trigger switch, such as the transition between pre-fill and fill states. Toggling a switch can also initiate a transition, as between idle and pre-injection states, and others.

The present configuration is also directed to software/firmware/hardware for accomplishing the methods discussed herein, and computer readable media storing software for accomplishing these methods. The various modules described herein can be accomplished on the same CPU, or can be accomplished on different CPUs. In compliance with the statute, the present configuration has been described in language more or less specific as to structural and methodical features. It is to be understood, however, that the present configuration is not limited to the specific features shown and described, since the means herein disclosed comprise forms of putting the present configuration into effect.

Methods can be, in whole or in part, implemented electronically. Signals representing actions taken by elements of the system and other disclosed configurations can travel over at least one live communications network. Control and data information can be electronically executed and stored on at least one computer-readable medium. The system can be implemented to execute on at least one computer node in at least one live communications network. Common forms of at least one computer-readable medium can include, for example, but not be limited to, a floppy disk, a flexible disk, a hard disk, magnetic tape, or any other magnetic medium, a compact disk read only memory or any other optical medium, punched cards, paper tape, or any other physical medium with patterns of holes, a random access memory, a programmable read only memory, and erasable programmable read only memory (EPROM), a Flash EPROM, or any other memory chip or cartridge, or any other medium from which a computer can read. Further, the at least one computer readable medium can contain graphs in any form, subject to appropriate licenses where necessary, including, but not limited to, Graphic Interchange Format (GIF), Joint Photographic Experts Group (JPEG), Portable Network Graphics (PNG), Scalable Vector Graphics (SVG), and Tagged Image File Format (TIFF).

While the present teachings have been described above in terms of specific configurations, it is to be understood that they are not limited to these disclosed configurations. Many modifications and other configurations will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

While the present teachings have been described in terms of specific configurations, it is to be understood that they are not limited to these disclosed configurations. Many modifications and other configurations will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

What is claimed is:

1. A apparatus to inject medical liquid comprising:
a housing;
a syringe barrel with a fitting at the closed end, the syringe attached to the housing;
a plunger including a lead screw, the plunger disposed in the syringe barrel
a mechanism to drive the lead screw toward the closed end of the syringe barrel;
an electric motor to drive the mechanism; and
wherein the mechanism is configured to be selectively disengaged from the lead screw and allowing the plunger to freely move axially in the syringe barrel.

2. The apparatus of claim 1 further comprising a second syringe that is configured to engage the fitting on the syringe barrel, wherein the second syringe may draw the plunger toward the closed end of the syringe barrel when the mechanism is selectively disengaged from the lead screw.

3. The apparatus of claim 1, wherein the lead screw has at least one flat surface parallel to the lead screw axis.

4. The apparatus of claim 3, the mechanism comprising:
a drive gear mechanically driven by the electric motor, the drive gear including an internal thread that engages the threads of the lead screw;
an anti-rotation flexure, the anti-rotation flexure have a first position wherein the lead
screw can not rotate and a second position wherein the lead screw is free to rotate; and wherein the rotation of the drive gear moves the lead screw along its axis when the anti-rotation flexure is in the first position and the lead screw can rotate and move long its axis when the drive gear is fixed and the anti-rotation flexure is in a second position.

5. The apparatus of claim 4, further comprising a key mounted in the housing, and the anti-rotation flexure comprises at least one arm that engage the at least one flat surface on the lead screw in the first position and the at least one arm extends to the key, wherein the key is configured to rotate in the housing and push the at least one arms away from the lead screw to put the anti-rotation flexure in the second position.

6. The apparatus of claim 4 wherein the anti-rotation flexure is configured to rotate about an axis perpendicular to the lead screw, wherein in the first position the anti-rotation flexure engages the at least one flat surface on the lead screw and is configured to rotate to a second position wherein the anti-rotation flexure does not touch the at least one flat surface of the lead screw.

7. The apparatus of claim 3, wherein the mechanism comprising:
a drive gear mechanically driven by the electric motor, the drive gear including a plurality of petal elements extending approximately on the axis of the of the lead screw, the petal elements have a thread surface that engages the lead screw in a first position and disengage from the lead screw in a second position.

8. The apparatus of claim 1, wherein the plunger further includes a tip with a distal face that contacts the liquid and proximal surface that is detachable connected to the distal end of the lead screw, wherein the tip detaches from the lead screw when a negative pressure is applied to distal face.

9. The apparatus of claim 1, wherein the plunger further includes a tip with a distal face that contacts the liquid and proximal surface that is configured to rotate relative to the lead screw.

10. The apparatus of claim 1, the mechanism comprising a drive gear mechanically driven by the electric motor, wherein the electric motor may be displaced to disengage from the drive gear whereby lead screw may freely turn the drive gear as it moves axially.

11. The apparatus of claim 1, further comprising a controller that receives input from a pressure sensor, at least one user input and controls the speed and direction of the electric motor.

12. The apparatus of claim 11, wherein the at least one user input includes a trigger and a direction switch.

13. The apparatus of claim 11 further comprising a battery connected to the controller.

14. The apparatus of claim 12 further comprising a battery switch

15. The apparatus of claim 14, further comprising:
a first circuit that is configured to be energized by the battery switch, the first circuit including a latch relay
a circuit that connect the battery to the controller through the latch relay, wherein the latch relay is closed by the first full pull of the trigger.

16. The apparatus of claim 1, further comprising status outputs comprising at least one of an LCD display, a vibratory element and one or more status lights.

17. The apparatus of claim 1, further comprising status outputs comprising an LCD display and a status light, wherein the status light and LCD displays are controlled by the controller.

18. The apparatus of claim 1, wherein the mechanism comprises a reduction gear train on the motor output shaft.

19. The apparatus of claim 18 further comprising a rotation sensor that detects the number of rotations of an element in the mechanism.

20. The apparatus of claim 18, where the rotation sensor measured rotations of a magnet on the end of the output shaft of the reduction gear train.

21. The apparatus of claim 1, wherein the apparatus is disposable.

22. The apparatus of claim 1, wherein the apparatus is only used for a single treatment.

23. The apparatus of claim 1, wherein the housing, the drive mechanism, the plunger, the electric motor and syringe barrel are disposable after a single treatment.

24. The apparatus of claim 1, wherein the housing, the drive mechanism, the plunger, the electric motor and syringe barrel are disposable are sterilized as a unit before use.
